(19) United States
(12) Patent Application Publication
(10) Pub. No.: US 2005/0070493 A1
(43) Pub. Date: Mar. 31, 2005

(54) Methods and compositions for treating Parkinson's disease

(76) Inventors: Stephen Eric Fawell, Framingham, MA (US); Orla Conneely, Houston, TX (US)

Correspondence Address: Patent Administrator;Biogen Idec Inc., 14 Cambridge Center, Cambridge, MA, 02142, US

(21) Appl. No.: 10/837,182

(22) Filed: Apr. 30, 2004

Related U.S. Application Data
(63) Continuation of application No. PCT/US02/34613, filed on Oct. 30, 2002.
(60) Provisional application No. 60/341,009, filed on Oct. 30, 2001.

(51) Int. Cl. A61K 48/00; C12N 15/85; C12N 5/08

(52) U.S. Cl. 514/44; 435/455; 435/368

(57) ABSTRACT

The present invention relates to novel methods and compositions for gene therapy. The invention also provides methods for treating diseases or disorders of the central nervous system associated with dopaminergic hypoactivity, disease, injury or chemical lesioning, including Parkinson's disease, manic depression, and schizophrenia.

### RELATED APPLICATIONS

This is a continuation of International Patent Application PCT/US02/34613 filed Oct. 30, 2002, which claims priority to U.S. Provisional Application No. 60/341,009, filed Oct. 30, 2001.

### BACKGROUND

Parkinson's disease (PD) is characterized by a progressive degeneration of dopaminergic neurons in the midbrain. While PD is a complex disorder of unknown etiology, it is postulated that symptoms manifest themselves after the fraction of functional dopaminergic cells falls below a threshold of twenty percent (Lange, K., et al., J. Neural Transm., 38:27-44). Symptoms include bradykinesia, akinesia, tremor, muscular rigidity, and postural instability (Duvoisin, R., (1993) Ann. N.Y. Acad. Sci., 648:187-193). The progressive loss of dopaminergic neurons is a hallmark of idiopathic (or sporadic) Parkinson's disease, is not prevented by current therapies (Latchman, D. S., et al., (2001) Rev. Neurosci. 12:69), and is thought to result from a combination of genetic predisposition (Vaughn, J. R., et al., (2001) Ann. Hum. Genet. 65:111), and environmental neurotoxic insult (Schapira, A. H., et al., (1998) Ann. Neurol. 44:S89; Shapira, A. H., (2001), Adv. Neurol. 86:155; Orth, M. et al., (2001) Am. J. Med. Genet. 106:27; Zhang, Y. et al., (2000) Neurol. Dis. 7:240). The recent identification of several genes associated with familial Parkinson's disease (Kitada, T., et al., (1998) Nature, 392:605; Lucking, C. B., et al., (2000), N. Engl. J. Med. 342:1560; Polymeropoulos, et al., (1997) Science 276:2045; Kruger, R., et al., (1998) Nature Genet., 18:106), has revealed a common causative link between defective ubiquitin proteasome mediated protein degradation pathways and the pathogenesis of the hereditary disease (Shimura, H. et al., (2001) Science 293:263; Leroy, E. et al., (1998) Nature 395:451; Tanaka, Y., et al., (2001) Hum. Mol. Gen. 10:919). Since the pathology associated with PD has been correlated with a depletion of dopamine and the progressive degeneration of dopaminergic neurons in the basal ganglia, the research efforts have focused on discovering means to prevent, protect and restore the nigrostriatal dopaminergic cell network.

In nerve cells, dopamine is synthesized from the amino acid tyrosine. Tyrosine is converted into dihydroxyphenylalanine (L-DOPA) by the enzyme tyrosine dehydroxylase (TH). This enzymatic activity of TH is the rate limiting step in dopamine biosynthesis. Subsequently, L-DOPA is converted to dopamine by the action of another enzyme, aromatic amino acid decarboxylase (AADC) (see, e.g., Elsworth, J. D., et al., (1997) Exp. Neurol., 144:4-9).

In the early stages of the disease, the rate limiting TH activity can be partially increased by oral administration of the dopamine precursor L-DOPA (Barbeau, A. (1961), Int. Congr: Series 38:152-153). While oral administration of L-DOPA is still the current therapy of choice, it is limited in its efficacy in multiple respects. In brief, this method of treatment is not site specific, resulting in unintended side effects (Harder, S., et al., (1995) Clin. Pharmacokinet. 29:243-256), it is difficult to maintain sustained levels of dopamine (Chase, T. N., et al., (1987) Adv. Neurol., 45:477-480), and, perhaps most importantly, this treatment only transiently eliminates symptoms and does not ultimately prevent the degeneration of dopaminergic cells (Malamed, E., et al., (1984) Advances in Neurology, 40:149-157). The problems associated with the administration of L-DOPA have been partially alleviated by newer pharmacologic treatments, but even the improved methods of treatment suffer from severe drawbacks. (for a review see, Hurtig, H. I., (1997) Exp. Neurol. 144:10-16). The dopamine agonists developed in the 1970's, Parlodel and Pergolide, resulted in fewer serious side effects but were also far less potent and therefore less effective. Deprenyl, a drug that decreases the rate of dopamine breakdown, showed limited efficacy in extended clinical trials. More recently, Sinemet, a combined regimen of L-DOPA and cardidopa, has been shown to minimize some of the side effects of L-DOPA, but still causes nausea, dyskinesia, psychosis, and hypotension. Overall, the efficacy of the current pharmacologic treatments is quite limited and the need for improved methods directed at the treatment of PD remains.

Since the blood brain barrier prevents many systematically administered drugs from entering into the central nervous system (CNS), the successful development of alternative drug delivery methods for Parkinson's disease has had limited success. L-DOPA and related pharmacologic agents are at least moderately effective at alleviating symptoms associated with PD because these molecules are able to cross the blood brain barrier. One alternative approach has focused on increasing the lipid content of polypeptides to facilitate their transport across the blood brain barrier (Gregoriadis, G., (1976), N. Engl. J. Med. 295:704-710). Another approach has concentrated on enhancing the permeability of capillaries in the brain (Saltzman, W. M., et al., (1991), Chem. Eng. Sci. 46:2429-2444).

Alternatively, one can avoid some of the complications posed by the blood brain barrier by administering the therapeutic agent directly into the CNS. Parkinson's disease is an attractive target for utilization of direct treatment strategies for several reasons. First, the observed neurodegeneration is selective to dopaminergic cells localized in the nigrostriatal cell network providing a circumscribed and limited target area. Second, the direct administration of therapeutic agents to a defined region would limit the adverse side effects observed with systemic drug delivery. Neural transplantation techniques offer one option for directed treatment, but these methods have yielded very preliminary and variable results (Freeman, T. B., (1997) Exp. Neurol., 144:47-50).

Accordingly, need remains for better and more effecacious PD treatments. To address the deficits in current treatment regimens for PD discussed above, we have developed an alternative treatment regimen based on methods and compositions of gene therapy. Gene therapy allows for the selective introduction of a functional gene that is either defective, down regulated, or whose function is impaired in some other manner. Gene therapy could similarly be used to introduce neuroprotective or neurorestorative nucleic acids into the CNS, or to increase the rate of production of a key protein, modulator or neurotransmitter. A succesful gene therapy method would overcome the limitations observed with pharmacologic treatments where the chronic administration of the current drugs results in progressively more severe side effects and debilitating motor complications (see e.g., Marsden, C., (1994) Clin. Neuropharmacol 17:S32-S44, Mouradian, M. M., et al., (1997) Exp. Neurol., 144:51-57). Since PD is associated with the depletion of dopamine and the loss of dopaminergic neurons, the physiological delivery of a gene critical in the biosynthesis of dopamine would stimulate dopamine production and alleviate the associated Parkinsonian symptoms. In the present invention the gene Nurr1, a nuclear transcription factor that plays a critical role in the differentiation and maintenance of dopaminergic cells, is administered into the CNS via gene therapy methods. In view of the limitations of current systemic therapies, gene delivery is a promising method for the treatment for CNS disorders such as PD.

### SUMMARY OF THE INVENTION

The present invention relates to novel methods for the protection and restoration of dopaminergic (DA) neuron function in the treatment of neuronal diseases. The invention teaches that expression of exogenous Nurr1 in neuronal cells, including, for example, cells of the substantia nigra (SN), results in enhanced survival of DA cell bodies and maintenance of the functional integrity of the nigrostriatal dopamine system. Accordingly, the disclosed methods are directed to methods of treating neuronal diseases, including, for example, Parkinson's disease, by administering a nucleic acid encoding Nurr1 to the subject.

In one aspect, the present invention provides methods for inhibiting the degeneration of catacholinergic neurons in a subject by providing an expression vector comprising a nucleic acid sequence encoding Nurr1 polypeptide and administering the expression vector to the brain of a subject under conditions that result in expression of Nurr1 and the prevention of the degeneration of catacholinergic neurons in the subject. In another aspect, the present inventions provides methods for treating a central nervous system disorder in a subject comprising providing an expression vector comprising a nucleic acid sequence encoding a Nurr1 polypeptide and administering the expression vector to neuronal cells of the subject under conditions that result in expression of Nurr1 in a therapeutically effective amount.

In one embodiment, a Nurr1 polypeptide is first produced in vitro and then administered to a subject in need thereof. In an exemplary embodiment, a nucleic acid encoding a Nurr1 polypeptide is administered in vivo to a subject in need thereof.

In one embodiment the invention provides methods where the expression vector is a viral vector. In another embodiment the viral vector is an adeno-associated viral vector or a recombinant adeno-associated viral vector. In yet another embodiment all the adeno-associated viral genes of the vector have been inactivated or deleted.

In one embodiment the expression vector is administered to the ventral midbrain. In another embodiment, the expression vector is administered to the substantia nigra. In yet another embodiment the expression vector is administered by stereotaxic injection.

In one embodiment the nucleic acid sequence encoding Nurr1 is operably linked to at least one transcriptional regulatory element. In another embodiment, the transcriptional regulatory element is a promoter sequence. In yet another embodiment the promoter is neuron specific. In yet another embodiment, the Nurr1 expression is either constitutive or regulatable.

In an exemplary embodiment, the subject is suffering from neuronal degeneration associated with one or more of the following: dopaminergic hypoactivity, disease, injury or chemical lesioning. In another embodiment the subject is suffering from neuronal disease. In yet another embodiment the neuronal disease is associated with a decrease in the level of dopamine. In yet another set of embodiments the neuronal disease is either Parkinson's, Schizophrenia or manic depression. In yet another embodiment, the catecholinergic neurons are dopaminergic. In yet another embodiment, the expression of Nurr1 causes an increase in tyrosine hydroxylase activity. In one embodiment the subject is human.

In certain embodiments, the treatment inhibits the degeneration of dopaminergic cells. In yet another embodiment the inhibition results from an increased production of dopamine.

In another aspect, the invention provides a neuronal cell transduced with a recombinant AAV virus comprising a nucleic acid encoding a Nurr1 polypeptide linked to at least one transcriptional element. In one embodiment the neuronal cell is dopaminergic. In another embodiment, the dopaminergic cell is in the substantia nigra. In yet another embodiment the neuronal cell is in situ. In yet another embodiment, the transcriptional element is a promoter or a neuron specific promoter.

In an exemplary embodiment the present invention provides methods for modulation of the levels of Nurr1 in the SN by gene therapy. This therapeutic approach permits intervention against progressive nigral DA neuron loss and for the functional recovery of the DA phenotype in patients suffering with Parkinson's disease.

### BRIEF DESCRIPTION OF THE FIGURES

FIG. 1. Nurr1 AS inhibits striatal DA content, TH activity and irTH of adult rats. Forty-eight hours after bilateral oligonucleotide infusion (2 nM, 1 μl), striatal tissue was dissected from rats and processed for DA content (panel a) and TH activity (panel b). In another set of animals, Nurr1 AS decreased TH immunostaining in the rat SNpc (panel c, representative animal) following unilateral SN infusion (right side of panel (n=3 rats per oligo)). For biochemical determinations, all samples were processed in triplicate and the experiments were repeated once. Data are expressed as mean±SEM concentrations and statistical differences were identified using Student's t-test (p<0.05).

FIG. 2. Unilateral Nurr1 AS induces a pattern of asymmetrical motor behavior. Time to initiation of first step by each limb (open bar-ipsilateral/right paw; closed bar-contralateral/left paw) was assessed (panel a). The length of step was determined by counting the total number of steps taken up a ramp by the animal and dividing it by the length of the ramp (panel b, open bars-RS, closed bars-AS). Data are presented as the mean±tSEM centimeters/step. Adjusting steps (panel c) were tested first in the forehand (dotted bars) and then in the backhand (hatched bars) direction. EBST (panel d, open bars-RS, closed bars-AS) was administered by handling the animal by its tail for 1 min. All behavioral testing commenced 48 h after oligonucleotide treatment and consisted of two tests per day. At the end of the second set of tests, apomorphine was given and testing was 30 min later. All experiments were repeated 14 d later. Oligonucleotide assignment was random and individuals blind to animal treatment conducted all tests.

FIG. 3. AAv.Nurr1 induces NBRE-CAT expression in CV 1 cells. Before injection into animals, AAv vectors were tested in cotransfection experiments using a CAT reporter gene under the control of the Nurr1 response element (NBRE-tk-CAT) in SKNSH neuroblastoma cells. The in vitro results are representative of 3 individual experiments with 2-3 replicates per experiment.

FIG. 4. AAv.Nurr1 rescues DA neurons from degeneration by 6-OHDA. On the left (panels a & c) are the representative control (uninfected, non-lesioned) sides of rat SN for each corresponding experimental (infected, lesioned or non-lesioned) side (panels b & d respectively). Rats were unilaterally infused in the striatum with 6-OHDA 28 d before tissue was processed for IHC (panels b & d). As described (25), experimental animals pretreated with 6-OHDA (panel d) were infected in the right SN with AAv.Nurr1 6 d later and tissue was processed at 28 d postlesioning. Experiments were repeated once. Arrows indicate injection site. Bar scale: 200 μm. Abbrev.: SN=substantia nigra, 3V=third ventricle.

FIG. 5. Percent of irTH cell loss in the right SN is less with postlesion AAv.Nurr1 treatment, an effect that is not associated with the virus itself. The data are presented as percent mean±tSEM of irTH cells counted in the treated ipsilateral SN versus the untreated contralateral SN. All animals were infected 7 days after right striatal 6-OHDA lesioning. The total number of TH-positive cells in 30 μm. sections was counted throughout each SN as described in the examples herein.

FIG. 6. SEQ ID NO:1 shows the nucleic acid sequence for rat Nurr1 (SEQ ID NO:1). GenBank Accession No. L08595.

FIG. 7. SEQ ID NO:2 shows the amino acid sequence of rat Nurr1 (SEQ ID NO:2).

FIG. 8. SEQ ID NO:3 shows the nucleic acid sequence of human Nurr1 (SEQ ID NO:3). GenBank Accession No. NM_{—}006186.

FIG. 9. SEQ ID NO:4 shows the amino acid sequence of human Nurr1 (SEQ ID NO:4).

### DETAILED DESCRIPTION OF THE INVENTION

1. General

In various aspects, the invention provides compositions and methods related to the regulation of Nurr1 expression as well as the treatment of neuronal diseases that may be associated with the depletion of catacholamines, including, for example, dopamine, norepinephrine and epinephrine (adrenaline). Also provided are neuronal cells comprising exogenous nucleic acid as well as methods and materials for inducing Nurr1 expression and treating central nervous system disorders associated with the degeneration of dopaminergic neurons. In an exemplary embodiment, the degeneration of dopaminergic neurons is caused by Parkinson's disease.

Cells expressing Nurr1 can be used, for example, to treat catecholamine-related deficiencies associated with disease states such as Parkinson's disease, manic depression, and schizophrenia. In particular, cells containing exogenous nucleic acid encoding Nurr1 are clinically useful, providing medical practitioners with biological material that can produce elevated levels of compounds such as DOPA, dopamine, and norepinephrine. In particular, cells containing exogenous Nurr1 nucleic acid will express Nurr1 polypeptide thus creating dopamine-producing cells that will be valuable in the medical treatment of dopamine-related deficiencies. For example, recombinant adeno-associated viral vectors containing exogenous Nurr1 nucleic acid may be administered to the substantia nigra region of a Parkinson's disease patient such that the production of dopamine is stimulated and the degeneration of dopaminergic neurons is prevented.

Nurr1 is a member of a ligand activated nuclear receptor superfamily and is a transcriptional activator localized predominantly in the brain, with a distribution that corresponds to dopamine containing cells. Nurr1 may be characterized by functional binding domains that promote transcription by binding to NGFI-B response elements (NBRE) located within the promoter region of the of tyrosine hydroxylase and the dopamine transporter genes. Nurr1 is essential for embryonic differentiation of midbrain dopaminergic (DA) neurons and its persistent expression in adult DA neurons suggests a role in their maintenance. Since the protection and restoration of dopamine function is critical for the treatment of neuronal diseases such as PD in which depletion of dopamine produces severe motor impairments, the administration of exogenous Nurr1 to the brain of PD patients offers a more efficacious method of treatment than provided by current methodologies.

2. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “AAV accessory function” refers generally to non AAV derived viral and/or cellular functions upon which AAV is dependent for its replication. Accessory functions may include, for example, non AAV proteins and RNAs that are required in AAV replication, including those involved in activation of AAV gene transcription, stage specific AAV mRNA splicing, AAV DNA replication, synthesis of Cap expression products and AAV capsid assembly. For example, viral-based accessory functions can be derived from any of the known helper viruses.

The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs may be used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as, for example, plasmids pAAV/Ad and pIM29+45 which encode both Rep and Cap expression products. See, e.g., Samulski et al. (1989) J. Virol. 63:3822-3828; and McCarty et al. (1991) J. Virol. 65:2936-2945. A number of other vectors have been described which encode Rep and/or Cap expression products. See, e.g., U.S. Pat. No. 5,139,941.

By “AAV cap coding region” is meant the art-recognized region of the AAV genome which encodes one or more of the capsid proteins VP1, VP2, and VP3, or functional homologues thereof. These Cap expression products supply the packaging functions which are collectively required for packaging the viral genome.

By “AAV rep coding region” is meant the region of the AAV genome which encodes one or more of the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N. (1992) Current Topics in Microbiol. and Immunol. 158:97-129; and Kotin, R. M. (1994) Human Gene Therapy 5:793-801. Suitable homologues of the AAV rep coding region include the human herpesvirus 6 (HHV-6) rep gene which is also known to mediate AAV-2 DNA replication (Thomson et al. (1994) Virology 204:304-311).

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, R. M. (1994) Human Gene Therapy 5:793-801; Bems, K. I. “Parvoviridae and their Replication” in Fundamental Virology, 2nd Edition, (B. N. Fields and D. M. Knipe, eds.) for the AAV-2 sequence. An “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAVX7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

The term “catecholamine” refers to a class of neurotransmitters including, for example, norepinephrine, epinephrine, dopamine, and functional analogs or derivatives thereof. “Catecholinergic” refers to neuronal cells that use catcholamines as their neurotransmitter.

The term “central nervous system” or “CNS” includes all cells and tissue of the brain and spinal cord of a vertebrate. Thus, the term includes, but is not limited to, neuronal cells, glial cells, astrocytes, cereobrospinal fluid (CSF), interstitial spaces, bone, cartilage and the like. The “cranial cavity” refers to the area underneath the skull (cranium). Regions of the CNS have been associated with various behaviors and/or functions. For example, the basal ganglia of the brain has been associated with motor functions, particularly voluntary movement. The basal ganglia is composed of six paired nuclei: the caudate nucleus, the putamen, the globus pallidus (or pallidum), the nucleus accumbens, the subthalamic nucleus and the substantia nigra. The caudate nucleus and putamen, although separated by the internal capsula, share cytoarchitechtonic, chemical and physiologic properties and are often referred to as the corpus striatum, or simply “the striatum.”

A “coding sequence” refers to a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence may be included downstream of (e.g., 3′ to) the coding sequence.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include:
(i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term DNA “control sequences” refers nucleotide sequences which facilitate replication, transcription and/or translation of a coding sequence. Exemplary control sequences include, for example, promoter sequences, polyadenylation signals, transcription termination sequences, upstream regulatory domains, origins of replication, internal ribosome entry sites (“IRES”), enhancers, and the like. Nucleic acid constructions of the invention may include one or more control sequences to facilitate replication, transcription, and/or translation in an appropriate host cell.

The term “degeneration” as used herein with reference to neuronal cells, refers to a deterioration in cell function and/or cell structure associated with injury, disease, and/or aging, and/or apoptosis associated with injury, disease, and/or aging, and/or necrosis associated with injury, disease, and/or aging. In exemplary embodiments, degeneration is associated with one or more of the following: disease (such as, for example, PD, schizophrenia, manic depression), a catecholamine deficiency, a dopamine deficiency, and chemical lesioning (e.g., via exposure to a neurotoxin such as 6-OHDA).

The term “dopamine” refers to a neurotransmitter having the chemical formula C₈H₁₁NO₂, and functional analogs or derivatives thereof. “Dopaminergic” refers to neuronal cells that use dopamine as their neurotransmitter.

An “effective amount” is an amount sufficient to effect beneficial or desired results. An effective amount can be administered in one or more administrations, applications or dosages.

The term “exogenous” refers to a nucleic acid or polypeptide present in a cell that does not naturally contain that nucleic acid or polypeptide. Non-naturally occurring nucleic acids are considered to be exogenous to a cell into which it has been introduced. In certain embodiments, non-naturally occurring nucleic acids may comprise nucleic acid sequences or fragments of nucleic acid sequences that are found in nature provided that the nucleic acid as a whole does not exist in nature. For example, a nucleic acid containing a genomic DNA sequence within an expression vector is considered to be a non-naturally occurring nucleic acid, and thus is considered to be exogenous to a cell once introduced into the cell, since that nucleic acid as a whole (genomic DNA plus vector DNA) does not exist in nature. Additionally, nucleic acids containing a promoter sequence and polypeptide-encoding sequence (e.g., cDNA or genomic DNA) in an arrangement not found in nature is considered to be a non-naturally occurring nucleic acid. In certain embodiments, a nucleic acid that is naturally occurring may be exogenous to a particular cell. For example, an entire chromosome isolated from a cell of person X would be considered an exogenous nucleic acid with respect to a cell of person Y once that chromosome is introduced into Y's cell.

A “gene” refers to a polynucleotide containing at least one open reading frame encoding a polypeptide. A gene may include intron sequences in addition to exon sequences.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention.

The term “host cell” denotes, for example, microorganisms, yeast cells, insect cells, and mammalian cells, that can be, or have been, used as recipients of an exogenous nucleic acid. The term includes the progeny of the original cell which has been transfected. The progeny of a single parental cell may not necessarily be completely identical in morphology or in genomic or total DNA complement as the original parent, due to natural, accidental, or deliberate mutation.

The term “isolated nucleic acid” refers to a polynucleotide of genomic, cDNA, or synthetic origin or some combination there of, which (1) is not associated with the cell in which the “isolated nucleic acid” is found in nature, or (2) is operably linked to a polynucleotide to which it is not linked in nature.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

“Non-human animals” of the invention include mammalians such as rodents, non-human primates, sheep, dog, cow, chickens, amphibians, reptiles, etc.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “Nurr1 nucleic acid” refers to a nucleic acid encoding a Nurr1 polypeptide, e.g., a nucleic acid comprising a sequence consisting of, or consisting essentially of, the polynucleotide sequence set forth in SEQ ID NO:1 or SEQ ID NO:3. A nucleic acid of the invention may comprise all, or a portion of: the nucleotide sequence of SEQ ID NO:1 or SEQ ID NO:3; a nucleotide sequence at least 60%, 70%, 80%, 90%, 95%, 96%, 97%, 98% or 99% identical to SEQ ID NO:1 or SEQ ID NO:3; a nucleotide sequence that hybridizes under stringent conditions to SEQ ID NO:1 or SEQ ID NO:3; nucleotide sequences encoding polypeptides that are functionally equivalent to polypeptides of the invention; nucleotide sequences encoding polypeptides at least about 60%, 70%, 80%, 85%, 90%, 95%, 98%, 99% homologous with an amino acid sequence of SEQ ID NO:2 or SEQ ID NO:4; nucleotide sequences encoding polypeptides having an activity of a polypeptide of the invention and having at least about 60%, 70%, 80%, 85%, 90%, 95%, 98%, 99% homology or more with SEQ ID NO:2 or SEQ ID NO:4; nucleotide sequences that differ by 1 to about 2, 3, 5, 7, 10, 15, 20, 30, 50, 75 or more nucleotide substitutions, additions or deletions, such as allelic variants, of SEQ ID NO:1 or SEQ ID NO:3; nucleic acids derived from and evolutionarily related to SEQ ID NO:1 or SEQ ID NO:3; and complements of, and nucleotide sequences resulting from the degeneracy of the genetic code, for all of the foregoing and other nucleic acids of the invention. Nucleic acids of the invention also include homologs, e.g., orthologs and paralogs, of SEQ ID NO:1 or SEQ ID NO:3 and also variants of SEQ ID NO:1 or SEQ ID NO:3 which have been codon optimized for expression in a particular organism (e.g., host cell).

The term “Nurr1 polypeptide” refers to polypeptides having the amino acid sequence set forth in SEQ ID NO:2 or SEQ ID NO:4 and functional equivalents thereof. In certain embodiments, a Nurr1 polypeptide refers to homologues, orthologues, paralogues, allelic variants, and alternative splice forms of SEQ ID NO:2 or SEQ ID NO:4 that retain at least one biologically activity of SEQ ID NO:2 or SEQ ID NO:4. In other embodiments, Nurr1 polypeptides include polypeptides comprising all or a portion of the amino acid sequence set forth in SEQ ID NO:2 or SEQ ID NO:4; the amino acid sequence set forth in SEQ ID NO:2 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to SEQ ID NO:2; and functional fragments thereof.

A nucleic acid is “operably linked” to another nucleic acid when it is placed into a functional relationship with another nucleic acid sequence. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence. For example, DNA encoding a presequence or secretory leader is operably linked to DNA encoding a polypeptide if it is expressed, for example, as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. In exemplary embodiments, operably linked sequences are contiguous and in the same reading phase. Linking may be accomplished, for example, by ligation at convenient restriction sites. If such sites do not exist, the synthetic oligonucleotide adaptors or linkers may be used in accordance with conventional practice.

The term “progenitor cell” as used herein refers to any cell that can give rise to a distinct cell lineage through cell division. In other words, progenitor cells can be generally described as cells that give rise to differentiated cells. For example, a neural progenitor cell is a parent cell that can give rise to a daughter cell having characteristics similar to a neural cell. The term “neural cell” as used herein refers to neurons, including dopaminergic neurons as well as glial cells, including astrocytes, oligodendrocytes, and microglia. For the purpose of this invention, all neuroepithelial cells of the diencephalon, telencephalon, mesencephalon, myelencephalon, and metencephalon as well as adult hippocampal progenitor cells (AHPs), adult subventicular zone stem cells, and adult spinal cord progenitor are considered to be neural progenitor cells. In addition, all neuroepithelial cells of the mesencephalon as well as AHPs, are considered to be midbrain neural progenitor cells. In certain embodiments, progenitor cells are mammalian cells that are derived from a mammal at any stage of development from blastula formation to adult.

As used herein, the term “promoter” means a DNA sequence that regulates expression of a selected DNA sequence operably linked to the promoter, and which effects expression of the selected DNA sequence in cells. The promoter is capable of binding RNA polymerase and initiating transcription of a downstream (3′-direction) coding sequence. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product.

The term “recombinant virion” refers to an infectious, replication-defective virus comprising a protein shell encapsidating a heterologous nucleotide sequence of interest. Recombinant virions may be produced in a suitable host cell having helper functions and/or accessory functions as needed for replication and packaging of the viral particles.

The term “recombinant virus” refers to a virus that has been genetically altered, e.g., by the substraction or addition or insertion of a heterologous nucleic acid construct into the particle.

The term “specifically hybridizes” refers to detectable and specific nucleic acid binding. Polynucleotides, oligonucleotides and nucleic acids of the invention selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

The terms “stringent conditions” or “stringent hybridization conditions” refer to conditions which promote specific hydribization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of the a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, N.Y.; and Tibanyenda, N. et al., Eur. J. Biochem. 139:19 (1984) and Ebel, S. et al., Biochem. 31:12083 (1992).

The terms “subject”, “individual” or “patient” are used interchangeably herein and refer to a vertebrate, preferably a mammal. Mammals include, but are not limited to, murines, simians, humans, farm animals, sport animals and pets.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a nucleotide integration vector and other nucleic acid molecules, into suitable host cells.

“Transcriptional regulatory element” is a generic term used throughout the specification to refer to DNA sequences, such as initiation signals, enhancers, and promoters, which induce or control transcription of protein coding sequences with which they are operably linked. In exemplary embodiments, transcription of a gene is under the control of a transcriptional regulatory sequence which controls the expression of the recombinant gene in a cell-type in which expression is intended. The recombinant gene may be under the control of transcriptional regulatory sequences which are the same or different from those sequences which control transcription of the naturally-occurring forms of the gene.

The term “treating” as used herein is intended to encompass curing as well as ameliorating at least one symptom of the condition or disease.

The term “vector” refers to a nucleic acid capable of transporting another nucleic acid to which it has been linked. One type of vector which may be used in accord with the invention is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto. Exemplary vectors, include, for example, plasmid, phage, transposon, cosmid, chromosome, virus, and virion.

The term “virion” refers to a complete virus particle, including a viral genome associated with a capsid protein coat.

3. Nurr1 Compositions.

Nurr1 Function

Nurr1, a member of the nuclear receptor superfamily of transcription factors (Law, S. et al., (1992) Mol. Endocrinol. 6:21-29; Tsai, J., et al., (1994) Annu. Rev. Biochem. 63:451), plays a critical role in embryonic differentiation of ventral midbrain DA neurons (Zetterstrom, R. H., et al., (1997) Science, 276:248; Castillo, S. O., et al., (1998) Mol. Cell Neurosci., 11:36). In the mouse, onset of Nurr1 expression in the ventral midbrain occurs at embryonic day 10.5 before the appearance of the DA marker enzyme, tyrosine hydroxylase (TH), at embryonic day 11.5. Nurr1-null mice lack midbrain dopaminergic neurons and die within 24 h after birth (Zetterstrom et al., Science 276:248-250 (1997); Saucedo-Cawdenas et al., Proc. Natl. Acad. Sci. USA 95:4013-4018 (1998); and Castillo et al., Mol. Cell. Neurosci. 11:36-46 (1998)). In addition, dopamine is absent in the substantia nigra and ventral tegmental area of Nurr 1-null mice (Castillo et al., Mol. Cell. Neurosci. 11:36-46 (1998)). However, TH immunoreactivity and mRNA expression in hypothalamic, olfactory, and lower brain stem regions were unaffected, and DOPA treatments, whether given to the pregnant dams or to the newborns, failed to rescue the Nurr1-null mice (Castillo et al., Mol. Cell. Neurosci. 11:36-46 (1998)). Ablation of Nurr1 results in embryonic developmental arrest of ventral midbrain DA precursor neurons and a lack of induction of a DA transmitter phenotype (Saucedo-Cardenas, O., et al., (1998) Proc. Natl. Acad. Sci. USA 95:4013). Further, DA precursor neurons fail to innervate their striatal target areas and die later by apoptosis (Saucedo-Cardenas, O., et al., (1998) Proc Natl. Acad. Sci. USA 95:4013; Wallen, A., et al (1999) Exp. Cell Res. 253:737).

Expression of Nurr1 continues in mature DA neurons during adulthood (Saucedo-Cardenas, O., et al., (1998) Proc Natl. Acad. Sci. USA 95:4013), suggesting that the protein may also play a role in normal functional maintenance of these neurons. Recent cell culture studies using in vitro transactivation assays demonstrate that Nurr1 can regulate transcription of select genes associated with the DA transmitter phenotype including those for TH and the dopamine transporter (Sakurada, K., et al., (1999) Develop. 126:4017; Sacchetti, P., et al., (2001) J. Neurochem. 76:1565). The mechanisms that influence survival of these neurons have important physiological and clinical significance for several reasons. First, the neurotransmitter dopamine plays a central role in control of voluntary movement, cognition, and emotive behaviors (Bjorklund, A., et al., (Amsterdam, 1984) Handbook of Chemical Neuroanatomy, Part 2:55-122). Next, disturbances in ventral midbrain DA neurons are implicated in motor control and their degeneration is associated with several neurologic and psychiatric diseases including Parkinson's disease. Lastly, current therapies for Parkinson's disease do not prevent the continuing degeneration of dopaminergic neurons.

Ectopic Nurr1 expression is sufficient to induce stem cells and neural precursors to adopt the dopaminergic cell fate (see, e.g., U.S. Pat. No. 6,284,539). Additionally, Nurr1 is believed to function at the later stages of dopaminergic cell diferentiation (Saucedo-Cardenas et al., (1998) P.N.A.S. 95(7):4013-8) and is thought to be essential for terminal differentation of dopamingergic neurons in the ventral midbrain (Witta et al., (2000) Brain Res Mol Brain Res 84(1-2):67-78).

Production of Nurr1

Nucleic acids encoding a Nurr1 polypeptide may be obtained using common molecular cloning or chemical nucleic acid synthesis procedures and techniques, including PCR. PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complimentary DNA strands.

General procedures for PCR are taught in MacPherson et al., PCR: A PRACTICAL APPROACH, (IRL Press at Oxford University Press, (1991)). PCR conditions for a given reaction may be empirically determined by one of ordinary skill in the art based on the teachings herein. A number of parameters influence the success of a reaction. Among these parameters are annealing temperature and time, extension time, Mg++ and ATP concentration, pH, and the relative concentration of primers, templates and deoxyribonucleotides. Exemplary primers are described below in the Examples. After amplification, the resulting fragments can be detected by agarose gel electrophoresis followed by visualization with ethidium bromide staining and ultraviolet illumination.

Another method for obtaining polynucleotides is by enzymatic digestion. For example, nucleotide sequences can be generated by digestion of appropriate vectors with suitable recognition restriction enzymes. The resulting fragments can then be ligated together as appropriate.

The polynucleotides used in the present invention may also be produced in part or in total by chemical synthesis, e.g., by the phosphoramidite method described by Beaucage and Carruthers, Tetra. Letts., 22:1859-1862 (1981) or the triester method according to the method described by Matteucci et al., J. Am. Chem. Soc., 103:3185 (1981), and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

Transcriptional Regulatory Elements

In certain embodiments, nucleic acids encoding a Nurr1 polypeptide may be operably linked to at least one transcriptional regulatory sequence. A variety of regulatory sequences are known in the art and may be selected to direct expression of the subject proteins in a desired fashion (time and place). Transcriptional regulatory sequences are described in Goeddel; Gene Expression Technology: Methods in Enzymology 185, Academic Press, San Diego, Calif. (1990).

In one embodiment, a Nurr1 nucleic acid may be operably linked to on eor more control elements that direct the transcription or expression of Nurr1 in the subject in vivo. Such control elements can comprise control sequences normally associated with Nurr1. Alternatively, heterologous control sequences can be employed. Useful heterologous control sequences may include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

In certain embodiments, a promoter may be a constitutive promoter, e.g., a strong viral promoter, such as, for example, a CMV promoter. The promoter can also be cell- or tissue-specific, that permits substantial transcription of the DNA only in predetermined cells, e.g., such as a promoter specific for fibroblasts, smooth muscle cells, or neuronal cells. A smooth muscle specific promoter is, e.g., the promoter of the smooth muscle cell marker SM22alpha (Akyura et al., (2000) Mol Med 6:983). For purposes of the present invention, both heterologous promoters and other control elements, such as CNS-specific and inducible promoters, enhancers and the like, may be used. Examples of heterologous promoters include the CMB promoter. Examples of CNS-specific promoters include those isolated from the genes from myelin basic protein (MBP), glial fibrillary acid protein (GFAP), and neuron specific enolase (NSE). Examples of inducible promoters include DNA responsive elements for ecdysone, tetracycline, hypoxia and aufin. A number of different viral and cellular promoters may be used to effectively direct and control transcription in rAAV vectors. In an exemplary embodiment the promoter is CMV, which is specific to the CNS and exhibits a preference for neurons over glial cells. (Baskar, J. F., et al., (1996), J. Virol. 70:3207-3214; Kaplitt, M. G., et al., (1994), Nat. Genet. 8:148-154; McCown, T. J., et al., (1996) Brain Res. 713:99-107). Neuron specific promoters include, but are not limited to, the PDGF B-chain promoter and the NSE promoter.

The promoter can also be an inducible promoter, e.g., a metallothionein promoter. Other inducible promoters include those that are controlled by the inducible binding, or activation, of a transcription factor, e.g., as described in U.S. Pat. Nos. 5,869,337 and 5,830,462 by Crabtree et al., describing small molecule inducible gene expression (a genetic switch); International patent applications PCT/US94/01617, PCT/US95/10591, PCT/US96/09948 and the like, as well as in other heterologous transcription systems such as those involving tetracyclin-based regulation reported by Bujard et al., generally referred to as an allosteric “off-switch” described by Gossen and Bujard (Proc. Natl. Acad. Sci. USA (1992) 89:5547) and in U.S. PAT. Nos. 5,464,758; 5,650,298; and 5,589,362 by Bujard et al. Other inducible transcription systems involve steroid or other hormone-based regulation.

The polynucleotide of the invention may also be introduced into the cell in which it is to be expressed together with another DNA sequence (which may be on the same or a different DNA molecule as the polynucleotide of the invention) coding for another agent. Exemplary agents are further described below. In one embodiment, the DNA encodes a polymerase for transcribing the DNA, and may comprise recognition sites for the polymerase and the injectable preparation may include an initial quantity of the polymerase.

In certain instances, a polynucleotide construct may permit translation for a limited period of time so that the polypeptide delivery is transitory. This can be achieved, e.g., by the use of an inducible promoter.

In an exemplary method of the invention, the DNA constructs are delivered using an expression vector. The expression vector may be a viral vector or a liposome that harbors the polynucleotide. Nonlimiting examples of viral vectors useful according to this aspect of the invention include lentivirus vectors, herpes simplex virus vectors, adenovirus vectors, adeno-associated virus vectors, various suitable retroviral vectors, pseudorabies virus vectors, alpha-herpes virus vectors, HIV-derived vectors, other neurotropic viral vectors and the like. A thorough review of viral vectors, particularly viral vectors suitable for modifying neural cells, and how to use such vectors in conjunction with the expression of polynucleotides of interest can be found in the book Viral Vectors: Gene Therapy and Neuroscience Applications Ed. Kaplitt and Loewy, Academic Press, San Diego, Calif., (1995). In brief, the transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are exemplary. The following additional guidance on the choice and use of viral vectors may be helpful to the practitioner. As described in greater detail below, such embodiments of the subject expression constructs are specifically contemplated for use in various in vivo and ex vivo gene therapy protocols.

4. Expression Vectors

Gene delivery vehicles useful in the practice of the present invention can be constructed, utilizing methodologies of molecular biology, virology, microbiology, molecular biology and recombinant DNA techniques, by one of skill in the art based on the teaching herein.

In certain embodiments, vectors for use according to the invention are expression vectors, i.e., vectors that allow expression of a nucleic acid in a cell. Expression vectors may contain both prokaryotic sequences, to facilitate the propagation of the vector in bacteria, and one or more eukaryotic sequences, such as transcription units that facilitate expression of a polypeptide in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papillomavirus (BPV-1), or Epstein-Barr virus (pHEBo, pREP-derived and p205) can be used for transient expression of proteins in eukaryotic cells. The various methods employed in the preparation of the plasmids and transformation of host organisms are known in the art. Other suitable expression systems for both prokaryotic and eukaryotic cells, as well as general recombinant procedures, may be found, for example, in Molecular Cloning A Laboratory Manual, 2^{nd} Ed., ed. by Sambrook, Fritsch and Maniatis (Cold Spring Harbor Laboratory Press: 1989) Chapters 16 and 17.

In other embodiments, viral vectors, including viral vectors suitable for modifying neural cells, amay be used in accordance with the invention (see, e.g., Viral Vectors: Gene Therapy and Neuroscience Applications Ed. Kaplitt and Loewy, Academic Press, San Diego, Calif., (1995). A transgene may be incorporated into any of a variety of viral vectors useful in gene therapy, such as recombinant retroviruses, adenovirus, adeno-associated virus (AAV), and herpes simplex virus-1. While various viral vectors may be used in the practice of this invention, AAV- and adenovirus-based approaches are of particular interest. Typically, viral vectors carrying transgenes are assembled from polynucleotides encoding the transgene(s), suitable regulatory elements and elements necessary for production of viral proteins which mediate cell transduction. In an exemplary embodiment, adeno-associated viral (AAV) vectors are employed.

Adeno-Associated Vectors

An exemplary viral vector system useful for delivery of the subject polynucleotides is the adeno-associated virus (AAV). Human adenoviruses are double-stranded DNA viruses which enter cells by receptor-mediated endocytosis. These viruses have been considered well suited for gene transfer because they are easy to grow and manipulate and they exhibit a broad host range in vivo and in vitro. Adenoviruses are able to infect quiescent as well as replicating target cells and persist extrachromosomally, rather than integrating into the host genome. AAV is a helper-dependent DNA parvovirus which belongs to the genus Dependovirus. AAV has no known pathologies and is incapable of replication without additional helper functions provided by another virus, such as an adenovirus, vaccinia or a herpes virus, for efficient replication and a productive life cycle. In the absence of the helper virus, AAV establishes a latent state by insertion of its genome into a host cell chromosome. Subsequent infection by a helper virus rescues the integrated copy which can then replicate to produce infectious viral progeny. The combination of the wild type AAV virus and the helper functions from either adenovirus or herpes virus generates a recombinant AVV (rAVV) that is capable of replication. One advantage of this system is its relative safety (For a review, see Xiao et al., (1997) Exp. Neurol. 144:113-124).

The AAV genome is composed of a linear, single-stranded DNA molecule which contains approximately 4681 bases (Berns and Bohenzky, (1987) Advances in Virus Research (Academic Press, Inc.) 32:243-307). The genome includes inverted terminal repeats (ITRs) at each end which function in cis as origins of DNA replication and as packaging signals for the virus. The internal nonrepeated portion of the genome includes two large open reading frames, known as the AAV rep and cap regions, respectively. These regions code for the viral proteins involved in replication and packaging of the virion. For a detailed description of the AAV genome, see, e.g., Muzyczka, N. (1992) Current Topics in Microbiol. and Immunol. 158:97-129.

Vectors containing as little as 300 base pairs of AAV can be packaged and can integrate. Space for exogenous DNA is limited to about 4.7 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector. AAV vectors are capable of transducing both dividing and non-dividing cells in vitro and in vivo (Afione, S. A., et al., (1996), J. Virol. 70:3235-3241; Flotte, T. R., et al., (1993), Pro. Natl. Acad. Sci USA 90: 10613-10617; Flotte, T., R., (1994), Am. J. Respir. Cell Mol. Biol. 11:517-521; Kaplitt, M. G., et al., (1994), Nat. Genet. 8:148-154; Kaplitt, M. G., et al., (1996), Ann. Thoracic Surg. 62:1669-1676; McCown, T. J., et al., (1996), Brain Res. 713:99-107; Muzyczka, N. (1992), Curr. Top. Microbiol. Immunol. 158: 97-129; Podsakoff, G., et al., (1994), J. Virol. 68: 5656-5666; Russell, D. W., et al., (1994), Proc. Natl. Acad. Sci USA 91:8915-8919; Ziao, X., et al., (1996), J. Virol., 70-:8098-8108). An example of a high frequency of successful integration of AAV DNA into non-dividing cells is the transduction of pulmonary epithelial cells (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973).

General methods for the construction and delivery of rAAV constructs are well known in the art and may be found in Barlett, J. S., et al., (1996), Protocols for Gene Transfer in Neuroscience; Towards Gene Therapy of Neurological Disorders, pp. 115-127. The AAV-based expression vector to be used typically includes the 145 nucleotide AAV inverted terminal repeats (ITRs) flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). A transgene incorporating the various Nurr1 DNA constructs of this invention can similarly be included in an AAV-based vector. As an alternative to inclusion of a constitutive promoter such as CMV to drive expression of the polynucleotide of interest, an AAV promoter can be used (ITR itself or AAV p5 (Flotte, et al. J. Biol. Chem. 268:3781-3790, 1993)).

AAV is also capable of infecting a broad variety of host cells including, primary neuronal and glial cells without triggering pathogenic or inflammatory side effects. (Wu et al., (1998) J. Virol. 72(7):5919-26; Xiao et al., (1997) Exp. Neurol. 144:113-124, W7, W21, W28). AAV has been used successfully to introduce gene constructs into neuronal cells in animals, including non-human primates. Sustained transduction of neuronal cells with rAAV vectors has been successfully demonstrated (Kaplitt, M. G., et al., (1994), Nat. Genet. 8:148-154). Effective transduction of neuronal cells in vitro by rAAV vectors has similarly been demonstrated (Flotte, T., R., (1994), Am. J. Respir. Cell Mol. Biol. 11:517-521; Podsakoff, G., et al., (1994), J. Virol. 68: 5656-5666; Russell, D. W., et al., (1994), Proc. Natl. Acad. Sci USA 91:8915-8919). The feasibility of use of rAAV vectors has already been tested in a number of in vivo systems including the brain (Alexander, I. E., et al., (1996), Hum. Gene Ther. 7:841-850; Doll, R. F., et al., (1996), Gene Ther. 3:437-447; During, M. J., et al., (1995), Soc. Neurosci. Abstr. 21:542; Kaplitt, J. G., et al., (1994) Nat. Genet 8:148-154; McCown, T. J., et al., (1996), Brain Res. 713: 99-107), spinal cord Kaplitt, J. G., et al., (1994) Nat. Genet 8:148-154) and muscle (Alexander I. E., et al., (1996), Hum Gene Ther: 7:841-850; Xiao, X., et al (1996), J. Virol., 70: 8098-8108). The published results from the in vivo studies are summarized in Table I in Xiao et al., (1997) Exp. Neurol. 144:113-124. For example, an AAV virus containing a gene encoding TH was administered by injection into the brain parenchyma of the monkey, which resulted in increased expression of TH in the monkey striatum (Bankiewicz et al., (1997) Exp. Neurol. 144:147-156).

Recombinant AAV (rAAV) has also been shown to succesfully transduce tissue targets in situ where gene expression has been maintained for periods of at least 18 months (Kaplitt, M. G., et al., (1996), Ann. Thorac. Surg., 62:1669-1676; McCown, T. J., et al., (1996), Brain Res. 713: 99-107). The feasibility of using the AAV virus in gene therapy is underscored by the fact that long term expression in neuronal cells has been demonstrated (Peel, A. L., et al., (1997), Gene Ther. 4:16-24) and AAV is already being tested in clinical trials (During, M., et al., (1996), Soc. Neurosci. Abstr. 18.12; Hermonat, P. L., and N. Muzyczka, (1984), Proc. Natl. Acad. Sci USA 81:6466-6470).

Production & Packaging of Adeno-Associated Vectors Carrying Nurr1

Polynucleotides may be inserted into vector genomes using methods known in the art based on the teachings herein. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA. Other means are known and available in the art.

In an exemplary embodiment, the viral vectors are AAV vectors. By an “AAV vector” is meant a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector typically includes at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

In one embodiment, AAV expression vectors are constructed using known techniques to provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and 3′) with functional AAV ITR sequences.

An AAV expression vector which harbors a Nurr1 DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1: 165-169; and Zhou et al. (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl.sub.2, 10 mM DTT, 33 μg/ml BSA, 10 mM-50 mM NaCl, and either 40 uM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

Additionally, heterologous genes can be produced synthetically to include AAV ITR sequences arranged 5′ and 3′ of one or more selected nucleic acid sequences. Preferred codons for expression of the chimeric gene sequence in mammalian CNS cells can be used. The complete heterologous sequence is assembled from overlapping oligonucleotides prepared by standard methods. See, e.g., Edge, Nature (1981) 292:756; Nambair et al. Science (1984) 223:1299; Jay et al. J. Biol. Chem. (1984) 259:6311.

Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

Production of rAAV Virions

A vector comprising transcriptional regulatory elements and the Nurr1 transgene of interest can be packaged into AAV virions. For example, a human cell line such as, for example, 293 can be co-transfected with the AAV-based expression vector and another plasmid containing open reading frames encoding AAV Rep and Cap genes under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the transgene) and expression of the viral capsid proteins. This system results in packaging of the transgene DNA into AAV virions (Carter, B. J., Current Opinion in Biotechnology 3:533-539, 1992; Kotin, R. M, Human Gene Therapy 5:793-801, 1994; Bartlett, J. S., et al., (1996), Towards Gene Therapy of Neurological Disorders, pp. 115-127; Flotte, T. R., et al., (1995), Gene Ther. 2:29-37; Samulski, R. J., et al., (1989), J. Virol. 63:3822-3828; Snyder, R., et al., (1996), Current Protocols in Human Genetics, pp 12.1.1-12.2.23). Typically, about three days after transfection, recombinant AAV is harvested from the cells along with adenovirus and the contaminating adenovirus is then inactivated by heat treatment. In another embodiment, packaging can be accomplished through the use of an engineered AAV packaging cell line and an AAV producer cell line where the AAV helper plasmid has been transfected into a human cell line (Clark, K. R., et al., (1995) Hum. Gene Ther. 6:1329-1341).

Methods to improve the titer of AAV can also be used to package the Nurr1 polynucleotide of the invention in an AAV virion. Such strategies include, but are not limited to: stable expression of the ITR-flanked transgene in a cell line followed by transfection with a second plasmid to direct viral packaging; use of a cell line that expresses AAV proteins inducibly, such as temperature-sensitive inducible expression or pharmacologically inducible expression. Alternatively, a cell can be transformed with a first AAV vector including a 5′ ITR, a 3′ ITR flanking a heterologous gene, and a second AAV vector which includes an inducible origin of replication, e.g., SV40 origin of replication, which is capable of being induced by an agent, such as the SV40 T antigen and which includes DNA sequences encoding the AAV rep and cap proteins. Upon induction by an agent, the second AAV vector may replicate to a high copy number, and thereby increased numbers of infectious AAV particles may be generated (see, e.g, U.S. Pat. No. 5,693,531 by Chiorini et al., issued Dec. 2, 1997). In yet another method for producing large amounts of recombinant AAV, a chimeric plasmid is used which incorporate the Epstein Barr Nuclear Antigen (EBNA) gene, the latent origin of replication of Epstein Barr virus (oriP) and an AAV genome. These plasmids are maintained as a multicopy extra-chromosomal elements in cells. Upon addition of wild-type helper functions, these cells will produce high amounts of recombinant AAV (U.S. Pat. No. 5,691,176 by Lebkowski et al., issued Nov. 25, 1997). In another system, an AAV packaging plasmid is provided that allows expression of the rep gene, wherein the p5 promoter, which normally controls rep expression, is replaced with a heterologous promoter (U.S. Pat. No. 5,658,776, by Flotte et al., issued Aug. 19, 1997). Additionally, one may increase the efficiency of AAV transduction by treating the cells with an agent that facilitates the conversion of the single stranded form to the double stranded form, as described in Wilson et al., WO96/39530.

AAV stocks can be produced as described in Hermonat and Muzyczka (1984) PNAS 81:6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in ORiordan et al., WO97/08298.

In order to produce rAAV virions, an AAV expression vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

For the purposes of the invention, suitable host cells for producing rAAV virions include microorganisms, yeast cells, insect cells, and mammalian cells, that can be, or have been, used as recipients of a heterologous DNA molecule. Cells from the stable human cell line, 293 (readily available through, e.g., the American Type Culture Collection under Accession Number ATCC CRL1573) are exemplary in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a convenient platform in which to produce rAAV virions.

AAV Helper Functions & AAV Accessory Functions

Host cells containing the above-described AAV expression vectors may be rendered capable of providing AAV helper functions to facilitate replication and encapsidation of the Nurr1 nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

AAV helper functions may be introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection.

Both AAV expression vectors and AAV helper constructs can be constructed to contain 20 one or more optional selectable markers. Suitable markers include genes which confer antibiotic resistance or sensitivity to, impart color to, or change the antigenic characteristics of those cells which have been transfected with a nucleic acid construct containing the selectable marker when the cells are grown in an appropriate selective medium. Exemplary selectable marker genes that are useful in the practice of the invention include, for example, the hygromycin B resistance gene (encoding Aminoglycoside phosphotranferase (APH)) that allows selection in mammalian cells by conferring resistance to G418 (available from Sigma, St. Louis, Mo.). Other suitable markers will be known to those of skill in the art based on the teachings herein.

In certain embodiments, the host cell (or packaging cell) may be rendered capable of providing non AAV derived functions, or “accessory functions,” in order to facilitate the production of rAAV virions. Particularly, accessory functions can be introduced into and then expressed in host cells using methods known to those of skill in the art. Commonly, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known agents. See, e.g., Buller et al. (1981) J. Virol. 40:241-247; McPherson et al. (1985) Virology 147:217-222; Schlehoferet al. (1986) Virology 152:110-117.

Alternatively, accessory functions can be provided using an accessory function vector. Accessory function vectors include nucleotide sequences that provide one or more accessory functions. An accessory function vector is capable of being introduced into a suitable host cell in order to support efficient AAV virion production in the host cell. Accessory function vectors can be in the form of a plasmid, phage, transposon or cosmid. Accessory vectors can also be in the form of one or more linearized DNA or RNA fragments which, when associated with the appropriate control elements and enzymes, can be transcribed or expressed in a host cell to provide accessory functions. See, for example, WO 97/17458.

Nucleic acid sequences providing the accessory functions can be obtained from natural sources, such as from the genome of an adenovirus particle, or constructed using recombinant or synthetic methods known in the art. In this regard, adenovirus-derived accessory functions have been widely studied, and a number of adenovirus genes involved in accessory functions have been identified and partially characterized. See, e.g., Carter, B. J. (1990) “Adeno-Associated Virus Helper Functions,” in CRC Handbook of Parvoviruses, vol. I (P. Tijssen, ed.), and Muzyczka, N. (1992) Curr. Topics. Microbiol. and Immun. 158:97-129. Specifically, early adenoviral gene regions E1a, E2a, E4, VAI RNA and, possibly, E1b are thought to participate in the accessory process. Janik et al. (1981) Proc. Natl. Acad. Sci. USA 78:1925-1929. Herpesvirus-derived accessory functions have been described. See, e.g., Young et al. (1979) Prog. Med. Virol. 25:113. Vaccinia virus-derived accessory functions have also been described. See, e.g., Carter, B. J. (1990), supra., Schlehofer et al. (1986) Virology 152:110-117.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as CsCl gradients. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile.

The resulting rAAV virions are then ready for use for DNA delivery to the CNS, including the cranial cavity of the subject.

rAAV Vector as a Non-Viral Delivery Vector

An alternative delivery option with rAAV vectors is to uncouple the integration episome properties from the viral component and to combine it with a non-viral delivery vehicle. In an exemplary embodiment the non-viral delivery vehicle is a liposome. (Baudard, M., et al., (1996), Hum. Gene Ther. 7:1309-1322; During, M., et al., (1996), Soc. Neurosci. Abstr. 18.12; Philip, R., et al., (1994), Mol. Cell. Biol. 14:2411-2418) Philip et al, have demonstrated the use of the rAAV-liposome combination in primary T-lymphocytes and primary and cultured tumor cells. (Philip, R., et al., (1994), Mol. Cell. Biol. 14:2411-2418). In that study, cell transfection resulted in sustained expression of the IL-2 gene. A similar methodology was also employed to in the treatment of Canavan's disease (During, M., et al., (1996), Soc. Neurosci. Abstr. 18.12). In vivo delivery of the rAAV-liposome combination has also been demonstrated. Baudard et al. have shown that sustained expression may be maintained in the mouse following an in vivo delivery of the complex through the tail of the mouse (Baudard, M., et al., (1996), Hum. Gene Ther. 7:1309-1322). In vivo delivery targeted to the central nervous system has been demonstrated by Wu et al., who achieved neuropeptide Y gene expression in the neocortex and the hypothalamic paraventricular nucleus of the brain following the injection of Sendai virosomes complexed with an rAAV plasmid. (Wu. P., et al., (1996) Gene Ther. 3:246-253).

For additional detailed guidance on AAV technology which may be useful in the practice of the subject invention, including methods and materials for the incorporation of a transgene, the propagation and purification of the recombinant AAV vector containing the transgene, and its use in transfecting cells and mammals, see e.g. Carter et al, U.S. Pat. No. 4,797,368 (10 Jan. 1989); Muzyczka et al, U.S. Pat. No. 5,139,941 (18 Aug. 1992); Lebkowski et al, U.S. Pat. No. 5,173,414 (22 Dec. 1992); Srivastava, U.S. Pat. No. 5,252,479 (12 Oct. 1993); Lebkowski et al, U.S. Pat. No. 5,354,678 (11 Oct. 1994); Shenk et al, U.S. Pat. No. 5,436,146(25 Jul. 1995); Chatterjee et al, U.S. Pat. No. 5,454,935 (12 Dec. 1995), Carter et al WO 93/24641 (published 9 Dec. 1993), and Natsoulis, U.S. Pat. No. 5,622,856 (Apr. 22, 1997). Further information regarding AAVs and the adenovirus or herpes helper functions required can be found in the following articles: Berns and Bohensky (1987), “Adeno-Associated Viruses: An Update”, Advanced in Virus Research, Academic Press, 33:243-306. The genome of AAV is described in Laughlin et al. (1983) “Cloning of infectious adeno-associated virus genomes in bacterial plasmids”, Gene, 23: 65-73. Expression of AAV is described in Beaton et al. (1989) “Expression from the Adeno-associated virus p5 and p19 promoters is negatively regulated in trans by the rep protein”, J. Virol., 63:4450-4454. Construction of rAAV is described in a number of publications: Tratschin et al. (1984) “Adeno-associated virus vector for high frequency integration, expression and rescue of genes in mammalian cells”, Mol. Cell. Biol., 4:2072-2081; Hermonat and Muzyczka (1984) “Use of adeno-associated virus as a mammalian DNA cloning vector: Transduction of neomycin resistance into mammalian tissue culture cells”, Proc. Natl. Acad. Sci. USA, 81:6466-6470; McLaughlin et al. (1988) “Adeno-associated virus general transduction vectors: Analysis of Proviral Structures”, J. Virol., 62:1963-1973; and Samulski et al. (1989) “Helper-free stocks of recombinant adeno-associated viruses: normal integration does not require viral gene expression”, J. Virol., 63:3822-3828. Cell lines that can be transformed by rAAV are those described in Lebkowski et al. (1988) “Adeno-associated virus: a vector system for efficient introduction and integration of DNA into a variety of mammalian cell types”, Mol. Cell. Biol., 8:3988-3996. “Producer” or “packaging” cell lines used in manufacturing recombinant retroviruses are described in Dougherty et al. (1989) J. Virol., 63:3209-3212; and Markowitz et al. (1988) J. Virol., 62:1120-1124.

Adenoviral Vectors

In certain embodiments, a viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. The infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage.

Recombinant adenovirus is capable of transducing both dividing and non-dividing cells. The ability to effectively transduce non-dividing cells makes adenovirus a good candidate for both in vivo and ex vivo gene transfer into neuronal cells. Adenoviruses have been demonstrated to be efficient in gene delivery to the central nervous system. Multiple examples of effective gene transfer into the CNS of non-human mammals using adenovirus have been demonstrated in the literature. (see Table II in Davidson et al., (1997) Exp. Neurol. 144:125-130). In particular, the efficacy of adenoviral mediated gene transfer has been demonstrated in the MPS VII and HPRT-deficiency mouse models. (Li, T., et al., (1995), Proc. Natl. Acad. Sci. USA 92:7700-7704; Plumb, T. J., et al., 1996), Neurosci. Lett. 214:159-162). Another set of references describe the successful delivery of a nucleic acid encoding the E. coli lacZ reporter gene into different regions of the brain with the use of the adenovirus vector. (Akli, S., et al., (1993), Nature Genet. 3:224-228; Bojocchi, G., et al., (1993), Nature Genet. 3:229-234; Davidson, et al., (1993), Nature Genet. 3:219-223; Le Gal La Salle, G., et al., (1993), Science 259:988-990). Other references describe the efficacy of adenovirus mediated gene transfer into the brain for the purpose of treating brain tumors. (Badie, B. K., et al., (1994), Neurosurgery 35:910-916; Colak, A., et al., (1995), Hum. Gene Ther. 6:1317-1322; Nilayer, G., et al., (1995), Proc. Natl. Acad. Sci. USA 92:9829-9833; Perez-Cruet, M. J., et al., (1994), J. Neurosci. Res. 39:506-511).

Adenovirus is particularly suitable for use as a gene transfer vector because of its mid-sized genome, ease of manipulation, high titer, wide target-cell range, and high infectivity. Both ends of the viral genome contain 100-200 base pair (bp) inverted terminal repeats (ITR), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan (1990) Radiotherap. Oncol. 19:197). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′ tripartite leader (TL) sequence which makes them exemplary mRNAs for translation.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584).

Adenovirus vectors have also been used in vaccine development (Grunhaus and Horwitz (1992) Siminar in Virology 3:237; Graham and Prevec (1992) Biotechnology 20:363). Experiments in administering recombinant adenovirus to different tissues include trachea instillation (Rosenfeld et al. (1991); Rosenfeld et al. (1992) Cell 68:143), muscle injection (Ragot et al. (1993) Nature 361:647), peripheral intravenous injection (Herz and Gerard (1993) Proc. Natl. Acad. Sci. USA 90:2812), and stereotactic inoculation into the brain (Le Gal La Salle et al. (1993) Science 254:988).

Furthermore, the virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers, e.g., 10⁹-10¹¹ plaque-forming unit (PFU)/ml, and they are highly infective. The life cycle of adenovirus does not require integration into the host cell genome. The foreign genes delivered by adenovirus vectors are episomal, and therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7, pp. 109-127). Expression of the inserted polynucleotide of the invention can be under control of, for example, the E1A promoter, the major late promoter (MLP) and associated leader sequences, the viral E3 promoter, or exogenously added promoter sequences.

In certain embodiments, the adenovirus vector may be replication defective, or conditionally defective. The adenovirus may be of any of the 42 different known serotypes or subgroups A-F. Adenovirus type 5 of subgroup C is the exemplary starting material in order to obtain the conditional replication-defective adenovirus vector for use in the method of the present invention. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the Nurr1 nucleic acid of interest at the position from which the E1 coding sequences have been removed. However, the position of insertion of the Nurr1 polynucleotide or construct of the invention in a region within the adenovirus sequences is not critical to the present invention. For example, it may also be inserted in lieu of the deleted E3 region in E3 replacement vectors as described previously by Karlsson et. al. (1986) or in the E4 region where a helper cell line or helper virus complements the E4 defect.

An exemplary helper cell line is 293 (ATCC Accession No. CRL1573). This helper cell line, also termed a “packaging cell line” was developed by Frank Graham (Graham et al. (1987) J. Gen. Virol. 36:59-72 and Graham (1977) J. General Virology 68:937-940) and provides E1A and E1B in trans. However, helper cell lines may also be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Adenoviruses can also be cell type specific, i.e., infect only restricted types of cells and/or express a transgene only in restricted types of cells. For example, the viruses may comprise a Nurr1 gene under the transcriptional control of a transcription initiation region specifically regulated by target host cells, as described e.g., in U.S. Pat. No. 5,698,443. Thus, expression of Nurr1 from replication competent adenoviruses can be restricted to certain cells by, e.g., inserting a cell specific response element to regulate synthesis of a protein necessary for replication, e.g., E1A or E1B.

DNA sequences of a number of adenovirus types are available from Genbank. For example, human adenovirus type 5 has GenBank Accession No.M73260. The adenovirus DNA sequences may be obtained from any of the 42 human adenovirus types currently identified. Various adenovirus strains are available from the American Type Culture Collection, Rockville, Md., or by request from a number of commercial and academic sources. A Nurr1 polynucleotide as described herein may be incorporated into any adenoviral vector and delivery protocol, by restriction digest, linker ligation or filling in of ends, and ligation.

Adenovirus producer cell lines can include one or more of the adenoviral genes E1, E2a, and E4 DNA sequence, for packaging adenovirus vectors in which one or more of these genes have been mutated or deleted are described, e.g., in PCT/US95/15947 (WO 96/18418) by Kadan et al.; PCT/US95/07341 (WO 95/346671) by Kovesdi et al.; PCT/FR94/00624 (WO94/28152) by Imler et al.; PCT/FR94/00851 (WO 95/02697) by Perrocaudet et al., PCT/US95/14793 (WO96/14061) by Wang et al.

Hybrid Adenovirus-AAV Vectors

In certain embodiments, a hybrid adenovirus-AAV vector may be used in accordance with the methods of the invention. Hybrid Adenovirus-AAV vectors comprise an adenovirus capsid containing a nucleic acid having a portion of an adenovirus, and 5′ and 3′ ITR sequences from an AAV which flank a selected transgene under the control of a promoter. See e.g. Wilson et al, International Patent Application Publication No. WO 96/13598. This hybrid vector is characterized by high titer transgene delivery to a host cell and the ability to stably integrate the transgene into the host cell chromosome in the presence of the rep gene. This virus is capable of infecting virtually all cell types (conferred by its adenovirus sequences) and stable long term transgene integration into the host cell genome (conferred by its AAV sequences).

The adenovirus nucleic acid sequences employed in this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353-end of the adenovirus, referred to as about map units 98.4-100).

The AAV sequences useful in the hybrid vector are viral sequences from which the rep and cap polypeptide encoding sequences are deleted and are usually the cis acting 5′ and 3′ ITR sequences. Thus, the AAV ITR sequences are flanked by the selected adenovirus sequences and the AAV ITR sequences themselves flank a selected transgene. The preparation of the hybrid vector is further described in detail in published PCT application entitled “Hybrid Adenovirus-AAV Virus and Method of Use Thereof”, WO 96/13598 by Wilson et al.

For additional detailed guidance on adenovirus and hybrid adenovirus-AAV technology which may be useful in the practice of the subject invention, including methods and materials for the incorporation of a transgene, the propagation and purification of recombinant virus containing the transgene, and its use in transfecting cells and mammals, see also Wilson et al, WO 94/28938, WO 96/13597 and WO 96/26285, and references cited therein.

Retroviruses

In certain embodiments, retroviral vectors may be used in accordance with the methods and compositions described herein. The retroviruses are a group of single-stranded RNA viruses characterized by an ability to convert their RNA to double-stranded DNA in infected cells by a process of reverse-transcription (Coffin (1990) Retroviriae and their Replication” In Fields, Knipe ed. Virology. New York: Raven Press). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsial proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin (1990), supra).

In order to construct a retroviral vector, a nucleic acid of interest, such as, for example, a Nurr1 nucleic acid, is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham:Butterworth; Temin, (1986) “Retrovirus Vectors for Gene Transfer: Efficient Integration into and Expression of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types.

The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a protein of the present invention, e.g., a transcriptional activator, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. An exemplary retroviral vector is a pSR MSVtkNeo (Muller et al. (1991) Mol. Cell Biol. 11:1785 and pSR MSV(XbaI) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in PCT/US96/09948 by Clackson et al. Examples of suitable packaging virus lines for preparing both ecotropic and amphotropic retroviral systems include Crip, Cre, 2 and Am.

Retroviruses, including lentiviruses, have been used to introduce a variety of genes into many different cell types, including neural cells, epithelial cells, retinal cells, endothelial cells, lymphocytes, myoblasts, hepatocytes, bone marrow cells, in vitro and/or in vivo (see for example, review by Federico (1999) Curr. Opin. Biotechnol. 10:448; Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g., single-chain antibody/env fusion proteins). This technique, while useful to limit or otherwise direct the infection to certain tissue types, and can also be used to convert an ecotropic vector in to an amphotropic vector.

Other Viral Systems

Other viral vector systems that can be used to deliver a Nurr1 nucleic acid of the invention may be derived from, for example, herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68: 1-10), and several RNA viruses. Exemplary viruses include, for example, an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al.(1990) J. Virol., 64:642-650).

Several defective HSV-1 vectors have been developed to deliver exogenous genes into the central nervous system. In the rat, the HSV-1 vector has been used to deliver either reporter genes or tyrosine hydroxylase genes to the brain by stereotaxic injection. (Bloom, D. C., et al., (1995), Mol Brain Res. 177:48-60; During, M. J., et al., (1994) Science 266:1399-1403; Fink, D. J., et al., (1992, Hum. Gene Ther. 3:12-19; Perez-Crut, J. J., et al., (1994), J. Neurosci. Res. 39:506-511; Wolfe, J. D., et al., (1992), Nature Genet. 1:379-384). In these experiments the genes of interest were shown to express in multiple brain regions in the areas near the injection site and in cells whose projections extend into the injection site. HSV-1 vectors have also been shown to induce stable and long term expression patterns. (Bloom, D. C., et al., (1995), Mol Brain Res. 177:48-60; During, M. J., et al., (1994) Science 266:1399-140). In an exemplary embodiment, HSV-based vectors are used to express Nurr1 in the substantia nigra of Parkinson's patients. A discussion of the application and clinical usefulness of HSV-based vectors to the treatment of Parkinson's disease may be found in Fink et al., (1997) Exp. Neurol. 144:103-112.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990, supra). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al. (1991) Hepatology, 14:124A).

5. Methods of Delivery

Any means for the introduction of polynucleotides into mammals, human or non-human, may be adapted to the practice of this invention for the delivery of the various constructs of the invention into the intended recipient. In an exemplary method of the invention, the DNA constructs are delivered using an expression vector. The expression vector may be a viral vector or a liposome that harbors the polynucleotide. Nonlimiting examples of viral vectors useful according to this aspect of the invention include lentivirus vectors, herpes simplex virus vectors, adenovirus vectors, adeno-associated virus vectors, various suitable retroviral vectors, pseudorabies virus vectors, alpha-herpes virus vectors, HIV-derived vectors, other neurotropic viral vectors and the like. The following additional guidance on the choice and use of viral vectors may be helpful to the practitioner. As described in greater detail below, such embodiments of the subject expression constructs are specifically contemplated for use in various in vivo and ex vivo gene therapy protocols. In another embodiment of the invention, the DNA constructs are delivered to cells by transfection, i.e., by delivery of “naked” DNA or in a complex with a colloidal dispersion system.

In vivo gene transfer provides another method for the direct delivery of therapeutic nucleic acids. There are several different gene delivery vehicles available for in vivo gene therapy. The methods include, but are not limited to, herpes simplex viral vectors (Federoff, H. J., et al., (1992), Proc. Natl. Acad. Sci USA 89:1636-1640; Geller, A. I., et al., (1988), Science 241:1667-1669; Geller, A. I, et al., (1990), Proc. Natl. Acad. Sci USA 87:1149-1153), adenoviral vectors (Caillaud, C., et al., (1993), Eur. J. Neurosci. 5:1287-1291; Chase, T. N., et al., (1987), Adv. Neurol. 45:477-480) lentiviral vectors (Naldini, L., (1996), Science 727:263-267), adeno-associated vectors (Muzyczka N., (1992) Immunol. 158:97-129; Samulski, R. J., et al., (1983), J. Virol. 63:3822-3828) and the transfer of naked DNA (Acsadi, G., et al., (1991), New Biol. 3:71-81; Jiao, S., et al., (1992), Hum. Gene Ther. 3:21-33; Wolff, J. A., et al., (1990), Science 247:1465-1468).

The polynucleotides of the invention may be operably linked to one or more transcriptional and translational regulation elements for injection as naked DNA into a subject. Schwartz et al., have demonstrated a successful transfer of naked DNA into the neuronal cells of the adult mouse. (Schwartz, B., et al., (1996), Gene Ther 3:405-411). Additionally, Wolff et al., have succeeded in the transducing muscle cells following the injection of naked DNA into muscle. (Wu, P., et al., (1996), Gene Ther 3:246-253). In an exemplary embodiment, the polynucleotide of the invention and necessary regulatory elements are present in a plasmid or vector. Thus, the polynucleotide of the invention may be DNA, which is itself non-replicating, but is inserted into a plasmid, which may further comprise a replicator. The DNA may be a sequence engineered so as not to integrate into the host cell genome.

Ex vivo gene therapy in the central nervous system compensates for the fact that neuronal cells do not readily regenerate. Ex vivo gene therapy allows for the option of replacing lost cells with transplanted cells expressing the gene of interest. Unfortunately, embryonic dopaminergic cells have poor survival rates. As an alternative, cultured cells may be engineered to manufacture either the dopamine neurotransmitter or a critical component of the dopamine biosynthesis pathway. Such cells are then grafted onto the affected region of the brain and the depleted neurotransmitter is replaced as the transplanted cells secrete dopamine or L-DOPA (Horellou, P., et al., (1990), Neuron 5:393-402; Horellou, P., et al., (1994), Proc. Natl. Acad. Sci USA 86:7233-7237; Horellou, P., et al., (1990) Eur. J. Neurosci. 2:116-119). This method has been shown to be successful in neuronal, endocrine and fibroblast cell lines. (Horellou, P., et al., (1990), Neuron 5:393-402; Horellou, P., et al., (1994), Proc. Natl. Acad. Sci USA 86:7233-7237; Horellou, P., et al., (1990) Eur. J. Neurosci. 2:116-119; Uchida, K., et al., (1970), Brain Res. 24:485-493; Wolff, J. A., et al., (1989), Proc. Natl. Acad. Sci. USA 86:9011-9014). Transplantation experiments may similarly be conducted with the use of cells derived from the central nervous system. Since such cells are derived from the brain, they have a high probability of successful integration into the central nervous system of the recipient. Another advantage of using cells derived from the central nervous system is the possibility decreasing immuno-resistance problems through the use of the patient's own cells in an autotransplantation procedure. Neural progenitor cells are another attractive cell type for this procedure. Use of immortalized neural progenitor cells in gene transfer transplantation experiments are described in Renfranz, P. J., et al., (1991), Cell 66:713-729; Onifer, S. M., et al., (1993), Exp. Neurol. 122:130-142; and Snyder, E. Y., et al., (1992), Cell 68:33-51. Any of the viral systems described below may be used to transduce the cells of interest prior to transplantation.

Multiple delivery approaches have been shown to be effective in the context of adenovirus delivery to the CNS. Methods include, but are not limited to, parenchymal delivery, intraventricular delivery and perivascular delivery (see Table I in Davidson et al., (1997) Exp. Neurol. 144: 125-130). Most often, intraparenchymal, intravitreal, subretinal, or ventricular injections have been used to effectively target the viral vector to the area of interest (Akli, S., et al., (1993), Nat. Genet. 3:224-228; Bajocchi, G., et al., (1993), Nat. Genet. 3:229-234; Davidson, B. L., et al., (1993), Nat. Genet. 3:219-223; Davidson, B. L., et al., (1994), Exp. Neurol. 125:258-267; Le Gal La Salle, G., et al., (1993), Science 259:988-990; Li, t., et al., (1994), Invest. Ophthalmol. Visual Sci. 35:2543-2549; Li, T., and G. L. Davidson, (1995), Proc. Natl. Acad. Sci USA 92:7700-7704; Plumb, T. J., et al., (1996), Neurosci. Lett. 214:159-162). Individuals skilled in the art with recognize that the methods described may be readily adapted to other viral vectors including retroviral vectors and adeno-associated vectors. In the exemplary embodiment the viral vector is an adeno-associated viral vector comprising a Nurr1 polypeptide.

Methods of delivery of viral vectors include, but are not limited to, intra-arterial, intra-muscular, intravenous, intranasal and oral routes. In an exemplary embodiment, rAAV virions may be introduced into cells of the CNS using either in vivo or in vitro transduction techniques. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant AAV virions with CNS cells e.g., in appropriate media, and screening for those cells harboring the DNA of interest can be screened using conventional techniques such as Southern blots and/or PCR, or by using selectable markers. Transduced cells can then be formulated into pharmaceutical compositions, described more fully below, and the composition introduced into the subject by various techniques, such as by grafting, intramuscular, intravenous, subcutaneous and intraperitoneal injection.

When a Nurr1 polynucleotide according to the invention is to be administered to the mammal directly, this may be accomplished via the direct injection of a vector including the polynucleotide, or an alternative delivery device, at a preselected target location in the brain of the mammal (see e.g., Kordower et al., (1998) Mov. Disorders 13:383-393; Freed et al., (1992) N.E.J. Med. 327:1549-1555; and Widner et al., (1992) N.E.J. Med 327:1556-1563. Preferably, the patient to be treated is placed in a stereotaxis frame to pinpoint the target site in the brain for injection (for a discussion of the method see Paxinos, The Rat Brain Stereotaxic Coordinates, 512.sup.nd Ed. Academic Press, San Diego, Calif., (1987). In an exemplary embodiment of the invention the preselected target location is a site in the mammal's substantia nigra. Following identification of a suitable site of injection to reach the preselected target location, a solution containing the polynucleotide of the invention is injected at a controlled rate. Control of the rate of injection is effected using methods known in the art (e.g., see Mandel et al., (1998) J. Neurosci. 18:4271-4284.

Pharmaceutical compositions will comprise sufficient genetic material to produce a therapeutically effective amount of the Nurr1 protein of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

As is apparent to those skilled in the art in view of the teachings of this specification, an effective amount of viral vector which can be added may be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

It should be understood that more than one transgene could be expressed by the delivered viral vector. Alternatively, separate vectors, each expressing one or more different transgenes, can also be delivered to the CNS as described herein. Furthermore, it is also intended that the viral vectors delivered by the methods of the present invention be combined with other suitable compositions and therapies. For instance, Parkinson's disease can be treated by co-administering an AAV vector expressing Nurr1 into the CNS and additional agents, such as dopamine precursors (e.g., L-dopa), inhibitors of dopamine synthesis (e.g. carbidopa), inhibitors of dopamine catabolism (e.g., MaOB inhibitors), dopamine agonists or antagonists can be administered prior or subsequent to or simultaneously with the vector encoding Nurr1. For example, L-dopa and, optionally, carbidopa, may be administered systemically.

Naked DNA & Liposomes

Any means for the introduction of polynucleotides into mammals, human or non-human, may be adapted to the practice of this invention for the delivery of the various constructs of the invention into the intended recipient. In one embodiment of the invention, the Nurr1 DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. An exemplary colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al. Colloidal dispersion systems.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

6. Methods of Treatment

The invention also provides methods for treating diseases or disorders of the central nervous system associated with dopaminergic hypoactivity, disease, injury or chemical lesioning, including Parkinson's disease, manic depression, and schizophrenia. In certain embodiments, the methods and compositions of the invention may be useful for the treatment of a variety of CNS disorders, including disorders that display a pathophysiology consistent with a hypoactivity of catecholinergic neurons. During development, neural stem cells differentiate into the different types of neurons and glia found in the adult central nervous system (CNS) and peripheral nervous system (PNS). In general, these different types of neurons are classified based on the particular types of neurotransmitters they produce. For example, dopaminergic neurons produce dopamine, while noradrenergic neurons produce norepinephrine. The neurotransmitters dopamine and norepinephrine belong to a class of compounds called catecholamines. A catecholamine is an ortho-dihydroxyphenylalkylamine that is derived from the common cellular metabolite tyrosine. For example, the catecholamines dopamine and norepinephrine are synthesized from tyrosine as follows: tyrosine is converted to dihydroxyphenylalamine (DOPA) by the enzyme tyrosine hydroxylase (TH), DOPA to dopamine by the enzyme aromatic L-amino acid decarboxylase (AADC), and dopamine to norepinephrine by the enzyme dopamine β-hydroxylase (DBH). The rate limiting step for both dopamine and norepinephrine synthesis is the conversion of tyrosine into DOPA by TH. In addition, dopamine can be converted to dihydroxyphenylacetic acid (DOPAC) by the enzymes monoamine oxidase (MAO) and aldehyde dehydrogenase.

According to certain embodiments of the invention, catecholamine-related deficiencies in a mammal (e.g., a human patient) can be treated by administering an effective amount of a Nurr1 nucleic acid. For example, the administration of an exogenous Nurr1 nucleic acid results in the induction of Nurr1 expression in the area of the brain where there is a catecholamine deficiency. In an exemplary embodiment, Nurr1 is administered to the substantia nigra of the brain.

In various embodiments, a catecholamine-related deficiency may be any physical or mental condition that is associated with or attributed to an abnormal level of a catecholamine such as dopamine or norepinephrine. This abnormal level of catecholamine can be restricted to a particular region of the mammal's brain (e.g., midbrain) or adrenal gland. A catecholamine-related deficiency can be associated with disease states such as Parkinson's disease, manic depression, and schizophrenia. In addition, catecholamine-related deficiencies can be identified using clinical diagnostic procedures.

In certain embodiments, a catecholamine-related deficiency may be treated by administering an exogenous Nurr1 nucleic acid to a cell of the mammal. The administration can be an in vivo, in vitro, or ex vivo administration as described herein. For example, an in vivo administration can involve administering a viral vector to the midbrain region of a mammal, while an ex vivo administration can involve extracting midbrain cells from a mammal, transfecting the cells with an exogenous nucleic acid in tissue culture, and then introducing the transfected cells back into the same mammal.

In one embodiment, induction of Nurr1 polypeptide expression in patients with catecholamine hypoactivity may stimulate tyrosine hydroxylase activity and the production of a depleted neurotransmitter. In an exemplary embodiment, the present invention is useful in the treatment of a CNS disease, such as, for example, Parkinson's disease. In another embodiment, the invention is useful in the treatment of dopaminergic hypoactvity induced by antipsychotics. Currently administered antipsychotic therapies are antidopaminergic and often cause Parkinsonian like symptoms in patients undergoing such treatments. The existing method of alleviating such symptoms consists of the administration of L-DOPA or other treatments for Parkinson's disease. In vivo induction of Nurr1 expression by methods described herein provides an alternative mechanism for the treatment of dopaminergic hypoactivity induced by antipsychotics. The methods disclosed herein for the induction of Nurr1 expression in the brain can also be used in the treatment of other CNS disorders affecting the catecholinergic system such as, for example, schizophrenia and manic depression.

Parkinson's Disease (PD) is characterized by loss of the nigrostriatal pathway and is responsive to treatments which facilitate dopaminergic transmission in the caudate-putamen. (Yahr and Bergmann, Parkinson's Disease (Raven Press, 1987), Yahr et al. (1969) Arch. Neurol. 21:343-54). The degeneration manifests itself in abnormal motor symptoms which include bradykinesia, postural abnormalities, rigidity and tremor. (R12).

The etiology of the Parkinson's disease is unkown, however experiments have implicated oxidative stress as a factor that contributes to the onset of Parkinson's disease. (for a review see e.g. Koutsilieri et al., (2002) J Neurol 249 Suppl 2: 1101-1105). Interestingly it has also been proposed that dopaminergic degeneration in Parkinson's disease progresses through the apoptotic pathway. (Von Coelln et al., (2001) J Neurochem 77(1):263-73). Von Coelln et al., have shown that adenovirus mediated induction of the XIAP protein can rescue 6-OHDA induced dopaminergic toxicity in cultured neuronal cells. ((2001) J Neurochem 77(1):263-73). Importantly, XIAP is a X-chromosomal inhibitor of apoptosis which functions by blocking caspase activation. Although this data is compelling, it should be noted that while the toxic effect of 6-OHDA is alleviated via caspase inhibition, the dopaminergic cells exhibit a loss of neurite outgrowth and a decrease in the rate of dopamine uptake. (Von Coelln et al., (2001) J Neurochem 77(1):263-73).

In the rat model for Parkinson's, animals are injected with a neurotoxin, 6-hydroxydopamine (6-OHDA), that is specific for catecholinergic cells. (Ungerstedt, U. et al., (1970) Brain Res 24:485-493). It is the model utilized in this invention as it is widely used to study the mechanisms of Parkinson's disease (Sauer et al., (1994) Neuroscience 59:401-15; Przedborski et al., (1995) Neuroscience 67:631-647) and neuronal cell death. (Ungerstedt et al., (1968) Eur. J. Pharmacol. 5:107-110). Unilateral 6-hydroxydopamine lesions of the substantia nigra are an established rodent model of PD. In this model, 6-OHDA selectively eliminates dopaminergic nerve terminals and eventually results in the degeneration of those neurons. The destructive actions of 6-OHDA are believed to be mediated by uptake through the dopamine transporter (Shimada et al., (1991) Science 254:576-578; Usdin et al., (1991) P.N.A.S. 88:11168-11171). Furthermore, reports that 6-OHDA has been detected in the brain and urine of PD patients (Andrew et al., (1993) Neurochem Res 18:1175-1177; Curtius et al., (1974) J Chromatogr 99:529-540) lead to the suggestion that 6-OHDA may be an endogenous neuotoxic factor in the pathogenesis of Parkinson's disease (Jellinger et al., (1995) J Neural Transm Suppl 46:297-314).

6-OHDA is readily oxidized to hydrogen peroxide, a highly reactive species, and is thought to exert its neurodegenerative effects on dopaminergic cells by initiating a free radical cascade. (Ferger et al., (2001) Neuroreport 12(6):1155-9) have shown evidence that 6-OHDA induced neuronal degeneration is progresses through the apoptotic cell death pathway. (Walkinshaw et al., (1994) Neuroscience 63(4):975-87; Lotharius et al., (1999) J Neurosci 19(4): 1284-93). Apoptosis is a term describing the process of cell “suicide” where a cell actively initiates its own destruction by activating an internal cascade of events. In a one embodiment of the present invention, a Nurr1 polypeptide is administered to a subject for the purpose of preventing apoptotic cell death in dopaminergic cells.

Although the cause is still under investigation, the pathophysiology is well documented in the literature. The loss of dopaminergic cells is thought to be the cause of the motor deficits associated with Parkinson's disease.

The practice of the present invention will employ, unless otherwise indicated, conventional methods of virology, microbiology, molecular biology and recombinant DNA techniques within the skill of the art. Such techniques are explained fully in the literature. See, e.g., Sambrook, et al. Molecular Cloning: A Laboratory Manual (Current Edition); Current Protocols in Molecular Biology (F. M. Ausubel, et al. eds., current edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.).

### EXEMPLIFICATION

The invention now being generally described, it will be more readily understood by reference to the following examples which are included merely for purposes of illustration of certain aspects and embodiments of the present invention, and are not intended to limit the invention in any way.

In the present study, inhibition of Nurr 1 expression by injection of Nurr 1 antisense oligonucleotides into the substantia nigra (SN) of adult rats resulted in a significant decrease of SN DA soma that was associated with a substantial reduction in striatal dopamine content and tyrosine hydroxylase (TH) activity. The biochemical changes induced by Nurr 1 AS resulted in the development of locomotor behavioral asymmetries analogous to those observed in Parkinson's disease. Importantly, DA neurons were partially rescued from degeneration when chemical lesioning with 6-hydroxydopamine (6-OHDA) in the striatum was followed by a single injection of a replication-defective adeno-associated (AAv) vector encoding Nurr1 into the SN. These results provide strong evidence that Nurr1 is essential for the survival of DA neurons in the SN and suggests that Nurr1 gene therapy may be a novel therapeutic intervention against the progressive nigral DA neuron loss associated with Parkinson's disease.

### Example 1

### The Biochemical Effect of Nurr1 Depletion in Adult Dopaminergic Neurons

To ascertain the biochemical effect of Nurr1 depletion in adult DA neurons, phosphorothiolated Nurr1 antisense (AS) oligonucleotides were administered to Sprague Dawley rats in two different experiments.

Briefly, rats were purchased (Harlan, St Louis Mo.), housed 2 per cage in the Baylor College of Medicine vivarium, maintained on a 12:12 h light:dark cycle (lights on at 0700 CST) with rat chow and water in excess ad libitum in accordance with Institutional and NIH Guidelines. After acclimation (7 days), animals underwent stereotaxic implantation of one or two cannulae (26 gauge, Plastics One, Roanoke Va.) using stereotaxic coordinates (G. Paxinos and C. Watson, The Rat Brain in Stereotaxic Coordinates. (Academic Press, Sydney, Australia, ed. 3, 1988)). The first cannula was placed into the substantia nigra (−5.3 mm anterior, 1.8 mm lateral, −7.4 mm ventral to the Bregma) and used for injection of oligonucleotides and AAv vectors. The second cannula was placed into the striatum (+1.0 mm anterior, 3.0 mm lateral, −5.0 mm ventral to the Bregma) and was used for injection of 6-OHDA.

Postoperatively, females were individually housed after surgery under conditions described above to avoid cannula disruption by cage-mates. For biochemical, immunohistochemical and behavioral studies, oligonucleotides were designed to the rat Nurr1 (GenBank accession No. L08595) (L M. Scearce, T. M. Laz, T. G. Hazel, L F. Lau, R. Taub, J. Biol. Chem. 268:885 (1993)). The sequence for AS was AAC ACA AGG CAT GGC TTC A (19 bp; aa 123-105) (SEQ ID NO:5) and RS was CAT TGA AGC GCT TGT TTC G (SEQ ID NO:6). In the behavioral studies, the results using the first set oligonucleotides were verified in an experiment using a second AS oligonucleotide (GAG GAC CCA TAC TGC G) (16 bp; aa 350-335) (SEQ ID NO:7) and a second RS oligonucleotide (CGC AGT ATG GGT CCT C) (SEQ ID NO:8). Synthetic, phosphorothiolated lyophilized oligonucleotides were dissolved in sterile distilled water within 30-60 min of administration. None of the oligonucleotides show homology with other reported genes in GenBank.

In the first experiment, rat Nurr1 antisense (AS) or random antisense (RS) oligonucleotides were given bilaterally into the SN. Two days later, the rats were euthansized and striatal tissues were collected for quantification of DA content and TH activity. For biochemical studies, animals (n=4 per treatment group) were euthansized under deep anesthesia, tissue was dissected using anatomical markers and approximately 20 mg of wet tissues was sonicated in 0.1 N perchloric acid. Following centrifugation at 12,000 rpm for 20 min, supernatants were collected for determination of DA and its metabolic products by high-performance chromatography (W.-D. Le, J. R. Bostwick, S. H. Appel, Dev. Brain Res. 67:375 (1992)). The values are expressed as ng/mg wet tissue. Striatal TH activity was assayed by coupling radiolabeled L-DOPA synthesis to its non-enzymatic decarboxylation (J. R. Bostwick and W.-D. Le Analytic Biochem. 192:125 (1991)). Briefly, rat striatum were homogenized in 50 mM Tris-HCl (pH 7.4) using a Teflon-glass homogenizer. Twenty-five μl aliquots of tissue homogenates were incubated in 96-well plate with a 16 μl substrate solution (¹⁴C-tyrosine and cofactors) for 20 min at 37° C. Thirty-three mM potassium ferricyanide was then added to the homogenate-substrate mixture to decarboxylate produced ¹⁴C-DOPA. ¹⁴Co2 released from each well after 45 min incubation at 55° C. was absorbed on overlying filter paper impregnated with hyamine-hydroxide, and quantified by radioisotope scintillation counting. The values are expressed as pmol/mg/20 min. Individuals blind to tissue treatment performed all assays.

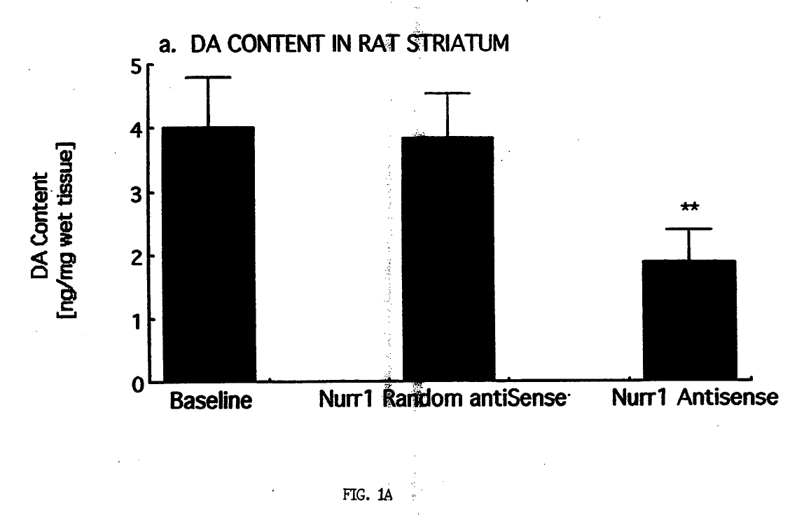

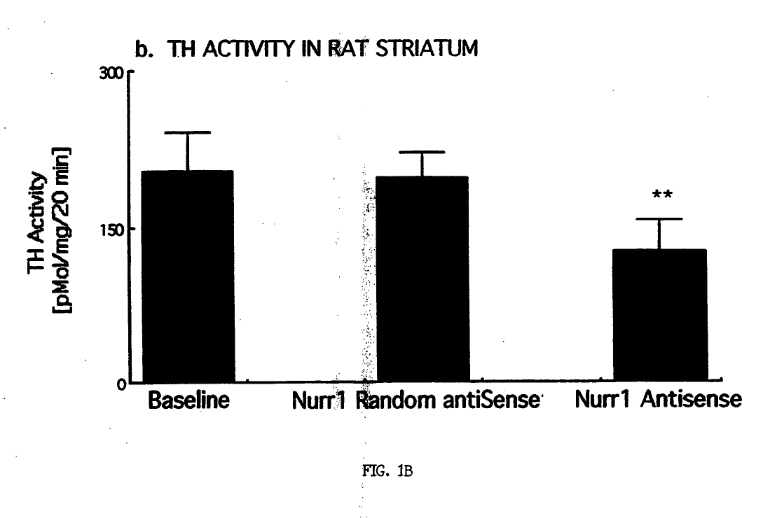

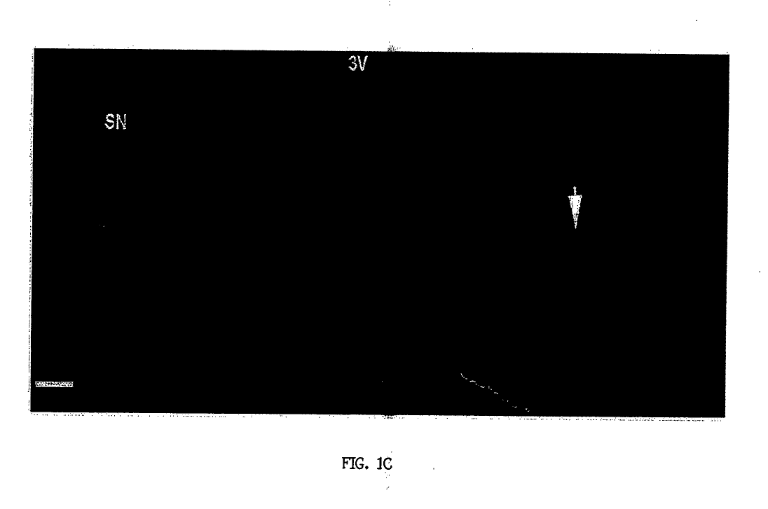

Nurr1 AS treatment significantly reduced both DA content (FIG. 1a) and TH activity (FIG. 1b) by approximately 52% and 39% respectively when compared with levels in control and RS oligonucleotide treated animals. In the second experiment, Nurr1 oligonucleotides were administered unilaterally into the SN. For immunohistochemistry (IHC) and cell counting, brains were postfixed in cold 4% PFA for 12 h, cryprotected with 30% sucrose in PBS, frozen in OCT, and cryosectioned. After blocking with a solution of 0.5% of normal goat serum, 0.1% Triton X100, and 0.05% sodium azide in PBS, 50 μm floating sections were incubated for 36 h at 4° C. with an 1:1000 dilution of the polyclonal anti-TH IgG (Protos Biotechnology, New York, N.Y.). After wash, a 1:250 dilution of Texas Red conjugated secondary antibody (Molecular Probes, CA) was added to track TH expressing cell bodies along the SN. After 24 h of reaction at 4° C., sections were washed and mounted on slides using VectaShield Kit (Vector Laboratories, CA). Sections were examined by fluorescence microscopy (Axiophot, Carl Zeiss), and photographed, followed by identification of the SN area for comparison. For unbiased cell counting in the oligo experiments, serial frozen sections (50 μm) were cut throughout the whole midbrain, adjacent sections around the injection site were processed for IHC, and irTH neurons in each SN of the seven adjacent sections were collated for analysis. For imaging in the rescue experiments, a biotinylated secondary antibody (1:800; Eugen International Inc. Allendale) and ABC reagent (Vector Labs, Burlingame) were used for detection. For unbiased cell counting, serial frozen sections (30 μm) were cut throughout the whole midbrain, resulting in 7 pairs from each animal. The total number of TH-positive cells was counted with a physical dissector as previously described (W.-D. Le, O. M. Conneely, Y. He, J. Jankovic, S. H. Appel, J. Neurochem. 73:2218 (1999)). Section pairs were termed “reference” and “adjacent”. Two adjacent sections were collected from every 3-section pair at 180 μm intervals and subjected to free-floating IHC. TH-positive neurons in the reference section but not recognized in the adjacent section at the same position were counted. Multiplying the number of TH-positive cells and the number of sections yielded the total of SN DA neurons.

Immunohistochemical analysis of the TH neurons in the SN (FIG. 1c) indicated that the decreased striatal DA content and TH activity were secondary to a reduction in the number of TH immunoreactive neurons in the AS injected side relative to uninjected side of the ventral midbrain. Finally, hematoxylin staining failed to detect individual cell toxicity at, or near, the injection sites following oligonucleotide treatment (data not shown). Hence, the data demonstrate that Nurr1 plays a critical role in maintaining biochemical function of nigrostriatal DA neurons in the adult.

### Example 2

### Nurr1 Depletion in Adult Dopaminergic Neurons Results in Significant Deficits in Motor Behavior

To determine whether the reduction in nigrostriatal DA transmission induced by Nurr1 AS was associated with locomotor abnormalities (Bjorklund, et al., (1984) Handbook of Chemical Neuroanatomy, eds. A. Bjorklund, et al.) Part 2:55-122 (Elsevier, Amsterdam)) behavioral analyses were undertaken following unilateral Nurr1 oligonucleotide treatment. Stainless steel cannulae were surgically implanted by stereotaxis in the dorsal SN on the animal's right side. During the subsequent 7 day recovery, the rats were familiarized with the examiner grip and ramp trained. Behavioral testing for baseline performance was conducted on the day before oligonucleotides were administered; testing for experimental effect was performed 2 days after oligonucleotides were given.

In all behavioral experiments, chronically cannulated rats (n=28) were injected unilaterally with oligonucleotides into the dorsal SN on the animal's right side following baseline testing. Behavioral testing commenced 48 h later. Thus, all animals served as their own controls. Pretest training was done 7-10 days after cannulation.

Stepping Test. Kinesis following Nurr1 AS treatment was tested using the stepping test as previously described (M. Olsson, M, G. Nikkhah, G, C. Bentlage, C, A. Bjorklund, J. Neurosci. 15:3863 (1995)) with revision. Briefly, the tests monitoring initiation time, stepping time, and step length were performed on a wooden ramp with a length of 31 inches connected to the rat's home cage. A smooth-surfaced table with a width of 29 inches was used for the test measuring adjusting steps. The stepping test comprised two parts. First, time to initiation of stepping by each forelimb, step length, and time required for the rat to cover the distance along the ramp with each forelimb was noted. Second, time to initiation of adjusting steps by each limb when the rat was moved sideways along the table surface was recorded. The examiner held the rats with one hand, fixing the hindlimbs and slightly raising the hind part above the surface. Stepping time was measured from initiation of movement until the rat reached home cage; step length was calculated by dividing the length of the ramp by the number of steps required for the rat to run up the ramp. Right paw testing preceded left paw testing during the first test, left preceded right during the second test.

Adjusting Steps. Adjusting steps were tested first in the forehand and then in the backhand direction. The number of adjusting steps was counted for both paws in the backhand and forehand directions of movements. For all parameters, the mean±SEM was calculated and statistical differences were identified using Student's t-Test (p<0.05).

Consistent with the moderate but not severe loss in striatal dopamine content observed after Nurr1 AS treatment, standard rotational testing failed to detect dysfunctional circling (data not shown). Therefore, more sensitive behavioral analyses for akinesis (stepping and elevated body swing) were undertaken to detect motor impairments. These tests correlate better with human condition than an automated rotational test since improvements in forelimb function (initiation and termination, switching motor strategies, and postural instability) and the motivational component of akinesia are thought to be more dependent on restoring DA to the SN.

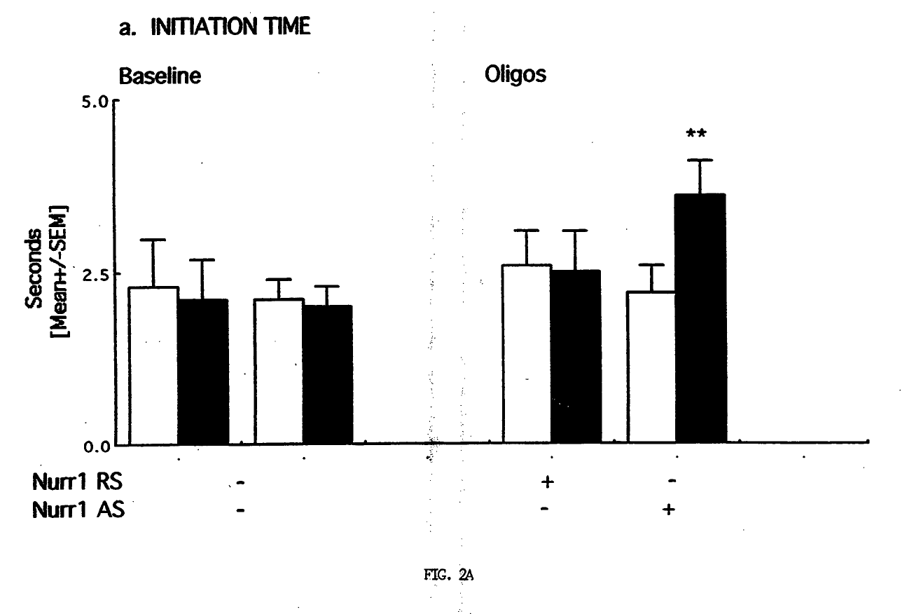

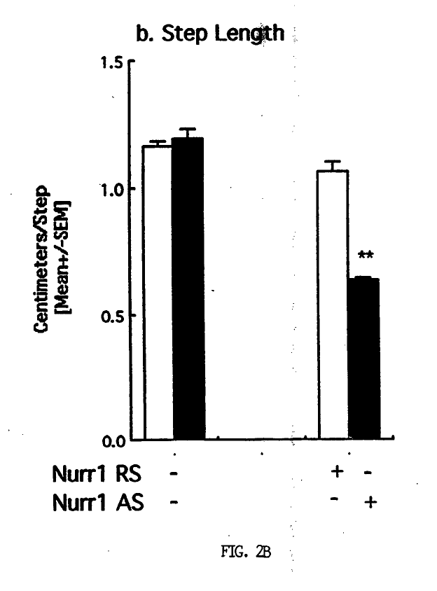

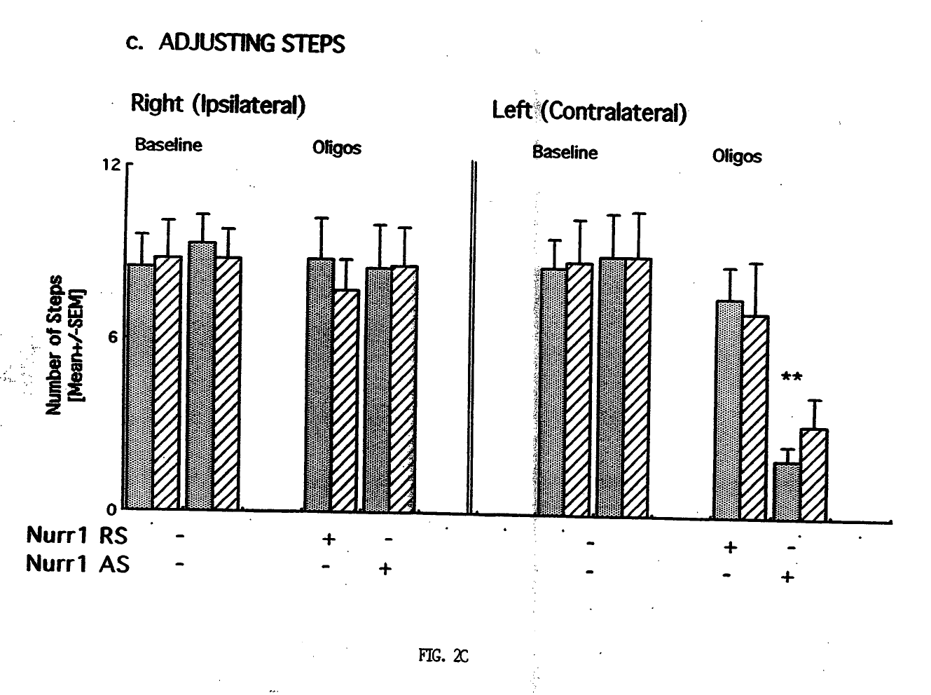

Nurr1 AS treatment to the right SN induced significant deficits in motor behavior. Delayed initiation of movement by the contralateral (lent) forelimb (FIG. 2a, closed bars) was significantly impaired 48 h after AS treatment compared with that of the ipsilateral (right) forelimb (open bars) after AS treatment and compared with pretreatment initiation times for both forelimbs. In the controls, RS treated animals exhibited comparable initiation times in both forelimbs regardless of pre- and post-treatment, demonstrating the absence of non-specific oligonucleotide effects on behavior. In addition to delayed initiation time, AS but not RS treatment resulted in a significant reduction in step length for walking up a ramp (FIG. 2b). More important, there was significant adjusting step impairment in contralateral paw performance (FIG. 2c, forehand, open bars; backhand, closed bars) following AS treatment whereas no impairment was observed in the ipsilateral paw, or either paw before oligonucleotide treatment or following RS treatment. Collectively, these findings are consistent with mild to moderate DA depletion in the right striatum and are similar to deficits observed almost immediately after 6-OHDA lesioning. Together, these findings are analogous to the akinetic impairments in gait and forelimb movements observed in patients with dopamine depletion.

The elevated body swing test (EBST) also was used to detect the effect of moderate DA depletion on asymmetrical motor behavior. It consists of measuring the frequency and direction of the swing behavior of the animal when held elevated by its tail for 1 min. Following collection of baseline rotational behavioral data, rats were divided into two treatment groups and received oligonucleotides (2 nM, in) composed of either RS or AS. Effect of oligonucleodde treatment was tested 48 h later. EBST was administered by simply handling the animals by its tail as previously described (C. V. Bodongan, P. R. Sanberg, J. Neurosci. 15, 5372 (1995)). The direction and overall total number of swings made by the animal over four consecutive 15 sec test periods were recorded, and the percentage of left and right swings per treatment were calculated. The criterion for biased swing behavior was set at 70% or higher. Statistical differences were tested for number of swings and percentages of total number of swings using Student's t-Test.

The results correlate with the location of moderate (>50%) to severe (>90%) lesions in the nigrostriatal DA pathway; e.g., SN lesioned animals exhibit fewer ipsilateral swings for a biased body swing contralateral to the lesioned side. The cannulated rats were pretested for symmetrical rotation and those with unbiased swing were injected with Nurr1 oligonucleotide into the unilateral SN. Experimental effect was assessed by ESBT 48 h later. Nurr1 AS treated animals exhibited fewer ipsilateral turns when elevated (FIG. 2d, open bars) than prior to treatment. The frequency of turning and direction of turning was unbiased in RS treated animals. Collectively, the data obtained from AS treated animals demonstrate that the acute loss of Nurr1 in adulthood produces biochemical and motor impairments that are consistent with those observed in animals that have undergone chemical lesioning of DA neurons with 6-OHDA and in patients with Parkinson's disease.

### Example 3

### Construction and Production and Bioactivity of an Adeno-associated Virus (AAV) Vector for Gene Transfer

Since Parkinson's disease is characterized by progressive degeneration of DA neurons in the SN that project to the striatum and since current therapies do not prevent this degeneration, restoration of DA neurons and their production of dopamine is a major therapeutic goal. To determine whether Nurr1 can rescue DA neurons from 6-OHDA induced degeneration, a replication-defective adeno-associated viral vector carrying the Nurr1 cDNA (AAv.Nurr1) under the control of the constitutively active cytomegalovirus (CMV) promoter was constructed.

For generation of AAv Vectors, both AAv vectors AAv-CMV-LacZ and AAv-CMV-Nurr1 were generated by triple transfection into 293 cells. The AAv cis-acting plasmid pAAv-CMV-LacZ was described previously (K. J. Fisher, et al., J. Virol. 70:520 (1996)). The cis-acting plasmid pAAv-CMV-Nurr1 was made by insertion of the Nurr1 gene downstream of the CMV promoter in a psub201-derived AAv plasmid lacking rep and cap. In each cis plasmid, the 5′ to 3′ organization was AAv ITR, CMV promoter, transgene, SV40 polyadenylation signal, AAv ITR. Virus was produced by triple transfection of pAAv-CMV-LacZ or pAAv-CMV-Nurr1 plus the rep and cap encoding plasmid pTrans-600 trans and the adenovirus helper function encoding plasmid pAdAF6 (Y. Zhang, N. Chirmule, G.-P. Gao, J. M. Wilson, J. Virol. 74:8003 (2000)) into 293 cells as described previously (X. Xiao, J. Li, R J. Samulski, J. Virol. 72:2224 (1988)). 293 cells were harvested 48 hours after transfection and frozen. Thawed cells were lysed by sonication. The lysate was treated with RNase A and DNase I, followed by deoxycholic acid treatment. AAv was purified by three sequential rounds of cesium chloride gradient ultracentrifugation and desalted as described previously (K. J. Fisher, et al., J. Virol. 70:520 (1996)). The AAv genome copy concentration was determined by real time quantitative PCR. Plasmids pAAv-CMV-LacZ, pTrans-600 trans and pAdΔF6 were obtained from of Drs. G.-P. Gao and J. M. Wilson (U. of Pennsylvania) (see, e.g., K. J. Fisher, et al., J. Virol. 70:520 (1996); Y. Zhang, N. Chirmule, G.-P. Gao, J. M. Wilson, J. Virol. 74:8003 (2000)). Prior to animal infection, bioreactivity of vectors was tested in cell culture. Initial infection of animals with the AAv.CMV-LacZ was used to determine effective viral dose levels and length of time required for effective expression.

The expression and transcriptional activity of Nurr1 from this vector were examined in SKNSH neuroblastoma cells. References describing the origin of SKNSH neuroblastoma cells, as well as the resources for where these cells may be obtained, are described on the following website: http://www.biotech.ist.unige.it/cldb/c14348.html. For example, SKNSH cells may be purchased from the ATCC American Type Culture Collection, at 10801 University Boulevard, Manassas, Va., 20110-2209. For cell culture and transfection, SKNSH neuroblastoma cells were seeded at a density of 10⁶ cells per 100-mm dish and 24 h thereafter infected with a 1 μl of a solution containing AAv.Nurr1 or AAv.LacZ (2.0×10¹² particles) in a total volume of 1000 μl DMEM/10% FCS. After 2-3 h, medium was replaced 3 ml of fresh DMEM/10% CFS for a 72 h incubation. Next, cells were cotransfected with a mixture of NBRE-CAT expression vector (1 μg/11) using Transfast (Fisher Scientific, Houston) per manufacturer's directions. NBRE-CAT plasmid contains the consensus response element for Nurr1 fused to the tk promoter and CAT gene. Medium was removed after 2 h and fresh DMEM/10% CFS medium was added. Thirty-six hours thereafter, cells were harvested and cell-free crude extracts were processed for protein determination by Bradford technique and CAT activity as described previously (E. Murphy, O. M. Conneely, Mol. Endocrinol. 11:39 (1997)).

Cotransfection of the vector with a Nurr1 responsive chlorampenicol acetyl transferase reporter gene (NBRE-CAT) into these cells confirmed that the viral expressed Nurr1 is transcriptionally active and resulted in a 9-fold stimulation of reporter gene expression relative to the basal activity observed using an AAv.LacZ control vector in place of AAv.Nurr1 (FIG. 3), thus confirming the bioactivity of the AAv.Nurr1 vector.

### Example 4

### In Vivo Delivery of AAv.Nurr1: Dosages and Methods

The question of whether Nurr1 can restore the DA phenotype in degenerated neurons was assessed in vivo using the striatal 6-OHDA progressive lesion rat model of Parkinson's disease. Sauer H., Oertel W. H. Neurosci. 59:401 (1994). In this experiment, the 6-OHDA lesion was carried out 7 days prior to a single injection of AAv.Nurr1 into the right SN of the rats. For experimental treatment for rescue of DA neurons by Nurr1, chronically cannulated rats (n=6) were injected at time of surgery with 1 μl of 6-OHDA (10 μg) into the dorsal right striatum over 5 min. Seven days later, 3 of the animals were injected with 1 μl of AAv.Nurr1 (2.1×10¹² particles per ml, over 30 min) in the right SN. An additional 3 rats were administered 1 μl of AAv.LacZ (4.8×10¹² particles per ml over 30 min) in the right SN in the absence of 6-OHDA as controls for viral effect.

Twenty-one days after AAv infection, all animals were transcardially perfused with 4% PFA and brain tissue was collected for processing. For immunohistochemistry (IHC) and cell counting, brains were postfixed in cold 4% PFA for 12 h, cryprotected with 30% sucrose in PBS, frozen in OCT, and cryosectioned. After blocking with a solution of 0.5% of normal goat serum, 0.1% Triton X100, and 0.05% sodium azide in PBS, 50 μm floating sections were incubated for 36 h at 4° C. with an 1:1000 dilution of the polyclonal anti-TH IgG (Protos Biotechnology, New York, N.Y.). After wash, a 1:250 dilution of Texas Red conjugated secondary antibody (Molecular Probes, CA) was added to track TH expressing cell bodies along the SN. After 24 h of reaction at 4° C., sections were washed and mounted on slides using VectaShield Kit (Vector Laboratories, CA). Sections were examined by fluorescence microscopy (Axiophot, Carl Zeiss), and photographed, followed by identification of the SN area for comparison. For unbiased cell counting in the oligo experiments, serial frozen sections (50 μm) were cut throughout the whole midbrain, adjacent sections around the injection site were processed for IHC, and irTH neurons in each SN of the seven adjacent sections were collated for analysis. For imaging in the rescue experiments, a biotinylated secondary antibody (1:800; Eugen International Inc. Allendale) and ABC reagent (Vector Labs, Burlingame) were used for detection. For unbiased cell counting, serial frozen sections (30 μm) were cut throughout the whole midbrain, resulting in 7 pairs from each animal. The total number of TH-positive cells was counted with a physical dissector as previously described (W.-D. Le, O. M. Conneely, Y. He, J. Jankovic, S. H. Appel, J. Neurochem. 73:2218 (1999)). Section pairs were termed “reference” and “adjacent”. Two adjacent sections were collected from every 3-section pair at 180 gm intervals and subjected to free-floating IHC. TH-positive neurons in the reference section but not recognized in the adjacent section at the same position were counted. Multiplying the number of TH-positive cells and the number of sections yielded the total of SN DA neurons.

Preliminary animals studies were performed to assess colocalization of AAv and endogenous TH, vector does for minimal host reaction, and optimal stability of in vivo infection and transgene expression.

### Example 5

### Protection and Restoration of Dopaminergic Function by the Application of the AAV Vector Carrying the Nurr1 Gene

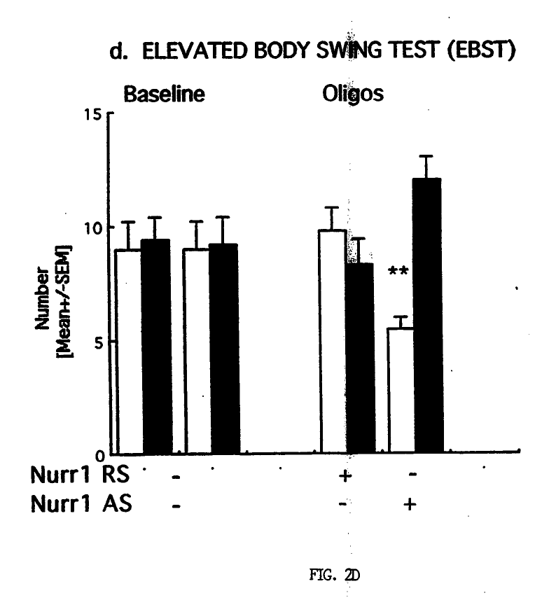

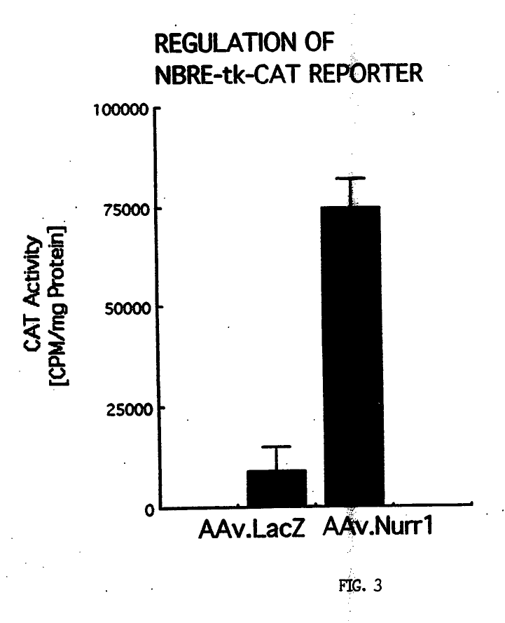

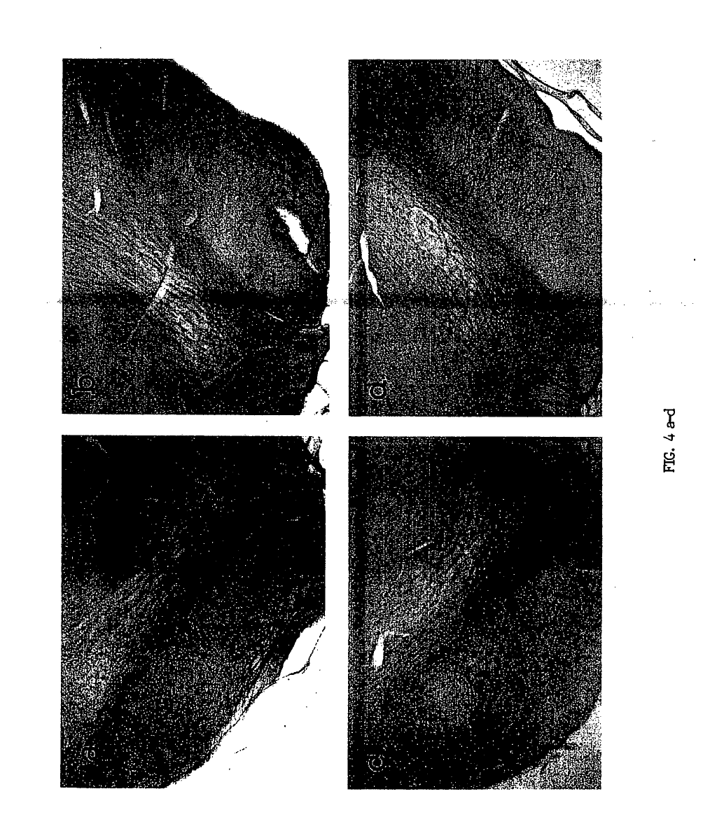

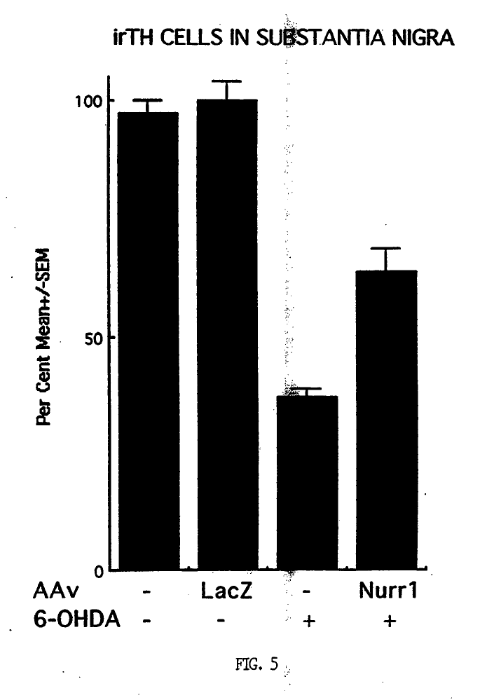

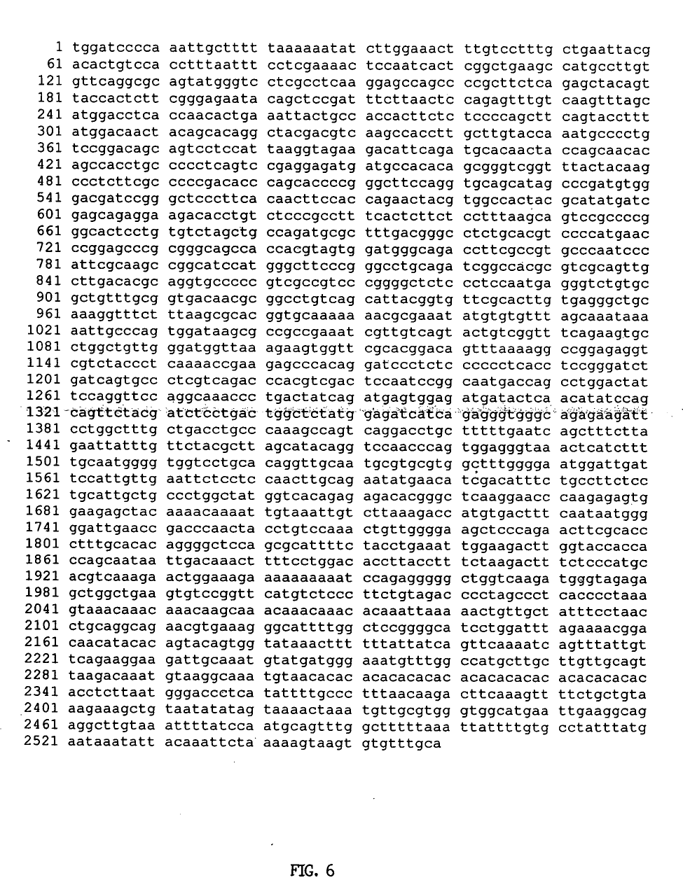

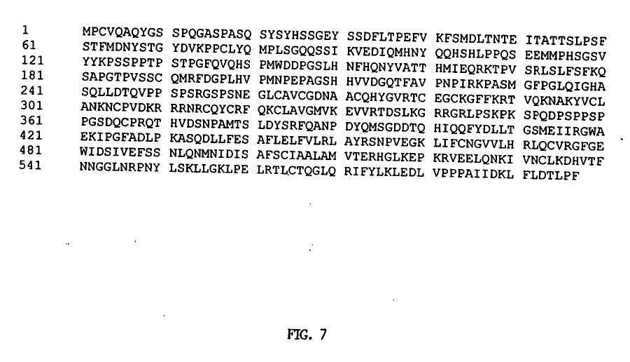

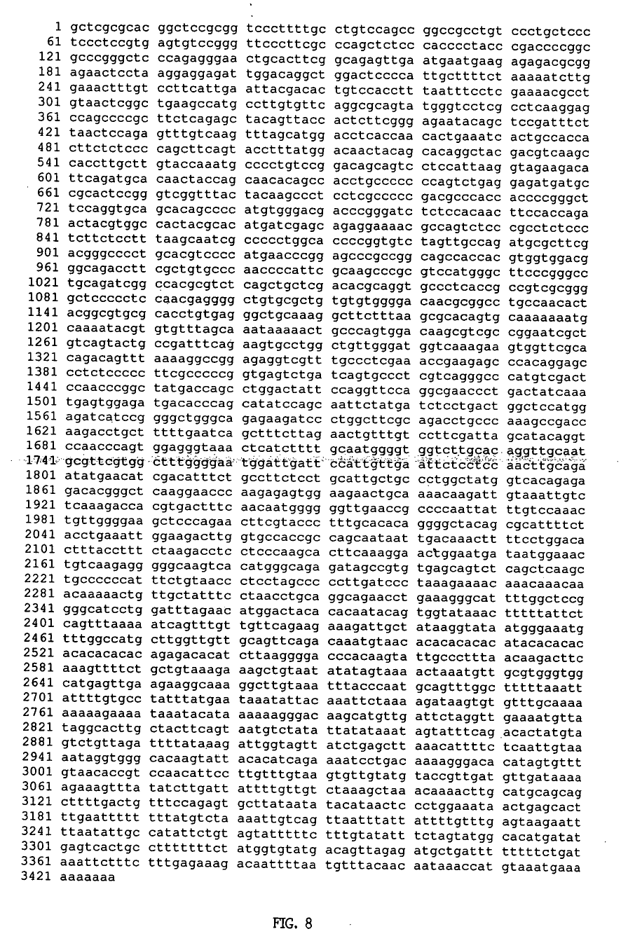

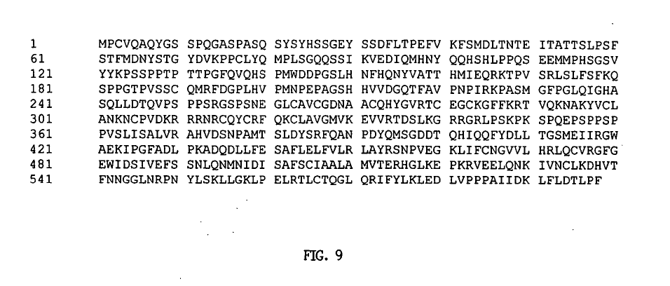

After 28 days, all rats were euthansized and tissue was processed for data analysis. In the control animals that underwent unilateral striatal 6-OHDA lesioning, abundant irTH cells were detected in the SN of the untreated side (FIG. 4a) whereas there was a significant loss of irTH positive cells (approximately 2115±290 cells) in the SNpc region of the 6-OHDA lesioned side (panel b). Significantly, in those experimental rats treated with a single injection of AAv.Nurr1 7 days after right striatal lesioning, (FIG. 4d) there was a marked increase in the number of irTH cells relative to that observed in the absence of virus (FIG. 4a). Indeed, approximately 3240±420 irTH positive cells were detected in the lesioned SN in those rats receiving AAv.Nurr1, thus providing a 43% restoration of irTH cells after a single injection of AAv.Nurr1 in the lesioned SN (FIG. 5). In those animals unilaterally treated with AAv.LacZ for 21 days, there was no significant effect of the virus on the number of irTH positive cells in the injected (5610±710 cells) versus non-injected (5373+489 cells) sides of the SN, confirming our preliminary data that also showed minimal host reaction to AAv infection. Hence, AAv.Nurr1 restored irTH expression in a significant number of neurons following chemical lesioning with 6-OHDA.

Equivalents

The present invention provides among other things novel methods and compositions for gene therapy applications. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: WO 99/10516, U.S. Pat. No. 6,312,949, U.S. Pat. No. 6,284,539, U.S. Pat. No. 6,180,613 and U.S. Pat. No. 6,309,634.

1. A method for inhibiting the degeneration of catacholinergic neurons in a subject, comprising:
(a) providing an expression vector comprising a nucleic acid sequence encoding a Nurr1 polypeptide; and
(b) administering said expression vector said subject under conditions that result in expression of Nurr1 in the brain, thereby preventing the degeneration of catacholinergic neurons in said subject.

2. The method of claim 1, wherein the catacholinergic neurons are dopaminergic.

3. The method of claim 1, wherein the subject is a human.

4. The method of claim 1, wherein the subject is suffering from neuronal degeneration associated with one or more of the following: dopaminergic hypoactivity, disease, injury, and chemical lesioning.

5. The method of claim 4, wherein said subject is suffering from a neuronal disease.

6. The method of claim 5, wherein the neuronal disease is associated with a decrease in the level of dopamine.

7. The method of claim 5, wherein the neuronal disease is Parkinson's disease.

8. The method of claim 5, wherein the neuronal disease is schizophrenia.

9. The method of claim 5, wherein the neuronal disease is manic depression.

10. The method of claim 1, wherein the expression vector is administered to the ventral midbrain.

11. The method of claim 10, wherein the expression vector is administered to the substantia nigra.

12. The method of claim 1, wherein the expression vector is administered by sterotaxic injection.

13. The method of claim 1, wherein the expression vector is a viral vector.

14. The method of claim 13, wherein the viral vector is an adeno-associated virus (AAV).

15. The method of claim 14, wherein the adeno-associated virus is a recombinant adeno-associated virus (rAAV).

16. The method of claim 15 wherein all adeno-associated viral genes of the vector have been inactivated or deleted.

17. The method of claim 1, wherein the nucleic acid sequence encoding a Nurr1 polypeptide is operably linked to at least one transcriptional regulatory element.

18. The method of claim 17 wherein said transcriptional regulatory element is a promoter sequence.

19. The method of claim 18 wherein said promoter sequence is a neuron specific promoter.

20. The method of claim 1, wherein Nurr1 expression is either constitutive or regulatable.

21. The method of claim 1, wherein the Nurr1 polypeptide comprises the amino acid sequence set forth in SEQ ID NO:2 or SEQ ID NO:4.

22. A method for treating a central nervous system disorder in a subject, comprising:
(a) providing an expression vector comprising a nucleic acid sequence encoding a Nurr1 polypeptide; and
(b) administering said expression vector to neuronal cells of said subject under conditions that result in expression of Nurr1 in a therapeutically effective amount.

23. A method of claim 22, wherein the expression vector is administered to the substantia nigra.

24. A method of claim 22, wherein the expression vector is administered in vivo.

25. A method of claim 22, wherein the expression vector is administered by sterotaxic injection.

26. A method of claim 22, wherein the expression vector is a viral vector.

27. A method of claim 26, wherein the viral vector is a recombinant adeno-associated virus (AAV).

28. A method of claim 22, wherein the nucleic acid sequence encoding a Nurr1 polypeptide is operably linked to at least one transcriptional regulatory element.

29. A method of claim 28, wherein said transcriptional regulatory element is a neuron specific promoter sequence.

30. A method of claim 22, wherein the central nervous system disorder is associated with a degeneration of dopaminergic cells.

31. A method of claim 30, wherein the degeneration of dopaminergic cells is associated with one or more of: dopaminergic hypoactivity, disease, injury and chemical lesioning.

32. The method of claim 22, wherein the central nervous system disorder is selected from the group consisting of Parkinson's disease, manic depression, and schizophrenia.

33. A method of claim 22, wherein said treatment inhibits the degeneration of dopaminergic cells.

34. A method of claim 33, wherein said inhibition results from the increased production of dopamine within said cells.

35. A method of claim 22, wherein expression of Nurr1 causes an increase in tyrosine hydroxylase activity.

36. A neuronal cell transduced with a recombinant AAV virus comprising a nucleic acid encoding a Nurr1 polypeptide linked to at least one transcriptional regulatory element.

37. A cell of claim 36, wherein said cell is a dopaminergic cell.

38. A dopaminergic cell of claim 37, wherein said dopaminergic cell is in the substantia nigra.

39. A cell of claim 36, wherein said cell is in situ.

40. A cell of claim 36, wherein said transcriptional regulatory element is a promoter.

41. A cell of claim 40, wherein said transcriptional regulatory element is a neuron-specific promoter sequence.

42. An AAV virus comprising a gene encoding a Nurr1 polypeptide operably linked to transcriptional and translational control elements.


SEQUENCE LISTING (8 sequences)

SEQ ID NO: 1 - DNA, 2559 bp, Rattus sp.
tggatccccaaattgctttttaaaaaatatcttggaaactttgtcctttgctgaattacgacactgtccacctttaatttcctcgaaaactccaatcactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcgggtcggtttactacaagccctcttcgcccccgacacccagcaccccgggcttccaggtgcagcatagcccgatgtgggacgatccgggctcccttcacaacttccaccagaactacgtggccactacgcatatgatcgagcagaggaagacacctgtctcccgcctttcactcttctcctttaagcagtccgccccgggcactcctgtgtctagctgccagatgcgctttgacgggcctctgcacgtccccatgaacccggagcccgcgggcagccaccacgtagtggatgggcagaccttcgccgtgcccaatcccattcgcaagccggcatccatgggcttcccgggcctgcagatcggccacgcgtcgcagttgcttgacacgcaggtgcccccgtcgccgtcccggggctctccctccaatgagggtctgtgcgctgtttgcggtgacaacgcggcctgtcagcattacggtgttcgcacttgtgagggctgcaaaggtttctttaagcgcacggtgcaaaaaaacgcgaaatatgtgtgtttagcaaataaaaattgcccagtggataagcgccgccgaaatcgttgtcagtactgtcggtttcagaagtgcctggctgttgggatggttaaagaagtggttcgcacggacagtttaaaaggccggagaggtcgtctaccctcaaaaccgaagagcccacaggatccctctcccccctcacctccgggatctgatcagtgccctcgtcagacccacgtcgactccaatccggcaatgaccagcctggactattccaggttccaggcaaaccctgactatcagatgagtggagatgatactcaacatatccagcagttctacgatctcctgactggctctatggagatcatcagagggtgggcagagaagattcctggctttgctgacctgcccaaagccagtcaggacctgctttttgaatcagctttcttagaattatttgttctacgcttagcatacaggtccaacccagtggagggtaaactcatcttttgcaatggggtggtcctgcacaggttgcaatgcgtgcgtggctttggggaatggattgattccattgttgaattctcctccaacttgcagaatatgaacatcgacatttctgccttctcctgcattgctgccctggctatggtcacagagagacacgggctcaaggaacccaagagagtggaagagctacaaaacaaaattgtaaattgtcttaaagaccatgtgactttcaataatgggggattgaaccgacccaactacctgtccaaactgttggggaagctcccagaacttcgcaccctttgcacacaggggctccagcgcattttctacctgaaattggaagacttggtaccaccaccagcaataattgacaaacttttcctggacaccttacctttctaagactttctcccatgcacgtcaaagaactggaaagaaaaaaaaaatccagagggggctggtcaagatgggtagagagctggctgaagtgtccggttcatgtctcccttctgtagacccctagccctcacccctaaagtaaacaaacaaacaagcaaacaaacaaacacaaattaaaaactgttgctatttcctaacctgcaggcagaacgtgaaagggcattttggctccggggcatcctggatttagaaaacggacaacatacacagtacagtggtataaacttttttattatcagttcaaaatcagtttattgttcagaaggaagattgcaaatgtatgatgggaaatgtttggccatgcttgcttgttgcagttaagacaaatgtaaggcaaatgtaacacacacacacacacacacacacacacacacacacacctcttaatgggaccctcatattttgccctttaacaagacttcaaagttttctgctgtaaagaaagctgtaatatatagtaaaactaaatgttgcgtgggtggcatgaattgaaggcagaggcttgtaaattttatccaatgcagtttggctttttaaattattttgtgcctatttatgaataaatattacaaattctaaaaagtaagtgtgtttgca

SEQ ID NO: 2 - protein, 597 aa, Rattus sp.
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPGFQVQHSPMWDDPGSLHNFHQNYVATTHMIEQRKTPVSRLSLFSFKQSAPGTPVSSCQMRFDGPLHVPMNPEPAGSHHVVDGQTFAVPNPIRKPASMGFPGLQIGHASQLLDTQVPPSPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPGSDQCPRQTHVDSNPAMTSLDYSRFQANPDYQMSGDDTQHIQQFYDLLTGSMEIIRGWAEKIPGFADLPKASQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 3 - DNA, 3427 bp, Homo sapiens
gctcgcgcacggctccgcggtcccttttgcctgtccagccggccgcctgtccctgctccctccctccgtgagtgtccgggttcccttcgcccagctctcccacccctacccgaccccggcgcccgggctcccagagggaactgcacttcggcagagttgaatgaatgaagagagacgcggagaactcctaaggaggagattggacaggctggactccccattgcttttctaaaaatcttggaaactttgtccttcattgaattacgacactgtccacctttaatttcctcgaaaacgcctgtaactcggctgaagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccaggttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataaggtataatgggaaatgtttggccatgcttggttgttgcagttcagacaaatgtaacacacacacacatacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgaaaaaaaaaa

SEQ ID NO: 4 - protein, 598 aa, Homo sapiens
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPTTPGFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQEPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 5 - DNA, 19 bp, Artificial Sequence
  Description of Artificial Sequence Synthetic oligonucleotide
aacacaaggcatggcttca

SEQ ID NO: 6 - DNA, 19 bp, Artificial Sequence
  Description of Artificial Sequence Synthetic oligonucleotide
cattgaagcgcttgtttcg

SEQ ID NO: 7 - DNA, 16 bp, Artificial Sequence
  Description of Artificial Sequence Synthetic oligonucleotide
gaggacccatactgcg

SEQ ID NO: 8 - DNA, 16 bp, Artificial Sequence
  Description of Artificial Sequence Synthetic oligonucleotide
cgcagtatgggtcctc